(19) United States
(12) Patent Application Publication
(10) Pub. No.: US 2026/0108656 A1
(43) Pub. Date: Apr. 23, 2026

(54) LEVAN-CATECHOL COMPOSITE, AND TISSUE ADHESION COMPOSITION AND NANOCLUSTER INCLUDING SAME

(71) Applicants: POSTECH RESEARCH AND BUSINESS DEVELOPMENT FOUNDATION, Pohang-si, Gyeongsangbuk-do (KR); Industry Academic Cooperation Foundation of Yeungnam University, Gyeongsan-si, Gyeongsangbuk-do (KR)

(72) Inventors: Dong Soo HWANG, Pohang-si (KR); Enhui LIN, Pohang-si (KR); Asila Ahmed Mohmme OSMAN, Pohang-si (KR); Jeong Hyun SEO, Gyeongsan-si (KR); Young Hoon SONG, Gyeongsan-si (KR)

(73) Assignees: POSTECH RESEARCH AND BUSINESS DEVELOPMENT FOUNDATION, Pohang-si, Gyeongsangbuk-do (KR); Industry Academic Cooperation Foundation of Yeungnam University, Gyeongsan-si, Gyeongsangbuk-do (KR)

(21) Appl. No.: 19/100,843

(22) PCT Filed: May 12, 2023
(86) PCT No.: PCT/KR2023/006486

§ 371 Date: Feb. 3, 2025

(30) Foreign Application Priority Data
Aug. 4, 2022 (KR) 10-2022-0097437
Apr. 14, 2023 (KR) 10-2023-0049539

(51) Int. Cl. A61L 26/00; A61K 47/69

(52) U.S. Cl. CPC A61L 26/0095; A61K 47/6929; A61L 26/0066

(57) ABSTRACT

The present invention relates to a levan-catechol composite, and a tissue adhesion composition and nanocluster, including same. In particular, the levan-catechol composite is prepared by the conjugation of levan and catechol and is applicable, by hydrogelation or nanoclustering thereof, for use in tissue adhesion in wet environments, wound healing, hemostasis, or drug delivery.

### CROSS REFERENCE TO RELATED APPLICATIONS

This application is a National Stage of International Application No. PCT/KR2023/006486 filed May 12, 2023, claiming priority based on Korean Patent Application No. 10-2022-0097437 filed Aug. 4, 2022 and Korean Patent Application No. 10-2023-0049539 filed Apr. 14, 2023, the entire disclosures of which are incorporated herein by reference.

### TECHNICAL FIELD

The present disclosure relates to a levan-catechol composite, a method for preparing the same, and a tissue adhesion composition and a nanocluster including the same.

### BACKGROUND ART

Tissue adhesives are rapidly gaining interest for tissue or wound closure applications due to many advantages over conventional closure techniques, including ease of application, strong adhesion, and effective sealing against air. However, commercially available tissue adhesive materials such as fibrin and cyanoacrylate, have limitations that include high toxicities, weak tissue adhesion, poor mechanical strengths in wet environments, and the like. Therefore, the development of new biocompatible and highly adhesive materials that overcome these limitations is an important objective.

Meanwhile, levan is a water-soluble biodegradable extracellular fructan containing a -(2,6)-linked backbone with occasional -(1,2) branches. The levan, a high molecular weight microorganism, may be commercially produced by Zymomonas mobilis when grown in a sucrose medium. The levan has remarkable properties, such as biodegradability and a lack of toxicity, and exhibits anti-tumor, anti-irritant and antioxidant behavior, as well as high cell-adhesion and proliferation performance. In addition, the levan plays a key role in metalloproteinase activation, which is a step involved in the healing of injured tissue. Moreover, the levan is one of the stickiest polysaccharides, which are ascribable to adhesion promoted by hydrogen bonding between its hydroxyl groups and the substrate, as well as its cohesive structure consisting of almost entirely flexible five-membered fructose residues that enable the formation of compact spheroids. Accordingly, the levan is a potential biomedical adhesive candidate. Despite these advantages, the levan has the major problem of having a low intrinsic viscosity and being diluted and washed away in wet environments. For example, since the adhesion of the levan to porcine skin decreases with decreasing concentration, water-resistant modifications are required for increasing adhesion under wet biomedical conditions.

Hydrogels are widely used in biomedicine due to their high water contents that are similar to the extracellular matrix (ECM). In addition, cross-linked network structures within the hydrogels serve to prevent the hydrogels from dissolving. Accordingly, the hydrogels are suitable wound closure candidates that accelerate the skin regeneration in human skin wounds by maintaining surface moisture. However, conventional hydrogels had the disadvantage of having poor mechanical properties, such as weak adhesion and attachment when bent, twisted, stretched, or compressed. Accordingly, various modifications and strategies have been recently reported to improve the mechanical properties of the hydrogels.

Marine mussels use proteinaceous thread-like adhesives (byssi) to strongly attach to various surfaces and protect themselves from wave-promoted dislodgement. Byssi is composed of 25 to 30 various mussel foot proteins (mfps), most of which contain 3,4-dihydroxyphenylalanine (DOPA) amino acid residues that play key roles in wet adhesion by forming covalent and noncovalent bonds to various inorganic/organic/metallic substrates in aqueous systems. Inspired by the mussel adhesion behavior, various DOPA-conjugated (or catechol-conjugated) natural polymers including hyaluronic acid and polyethylene glycol have been reported, but to date, there has been no report on the conjugation of levan and catechol or its use in wound healing.

Accordingly, the present inventors have completed the present disclosure by preparing a levan-catechol composite by the conjugation of levan and catechol, and by hydrogelating or nanoclustering the same, for use in tissue adhesion in wet environments, wound healing, hemostasis, or drug delivery.

### DISCLOSURE

### Technical Problem

An object of the present disclosure is intended to be applied for use in tissue adhesion in wet environments, wound healing, hemostasis, or drug delivery by preparing a levan-catechol composite by the conjugation of levan and catechol, and by hydrogelating or nanoclustering the same.

Another object of the present disclosure is to provide a tissue adhesion method, including treating a tissue adhesion composition including a levan-catechol composite according to the present disclosure, or a tissue adhesion composition including nanocluster according to the present disclosure, to a subject in need thereof.

Meanwhile, the technical objects to be achieved in the present disclosure are not limited to the aforementioned technical objects, and other technical objects, which are not mentioned above, will be apparently understood to a person having ordinary skill in the art from the following description.

### Technical Solution

To achieve the above-mentioned object, an aspect of the present disclosure provides a levan-catechol composite including carboxymethylated levan and dopamine.

The carboxymethylated levan may include a repeating unit represented by the following Chemical Formula 1.

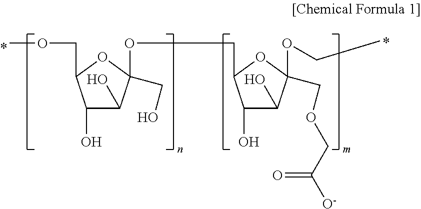

In Chemical Formula 1,

The n and m are mole fractions within the repeating unit, respectively, in which 0.7≤n≤0.95, 0.05≤m≤0.3, and n+m=1.

The levan-catechol composite may include a repeating unit represented by the following Chemical Formula 2.

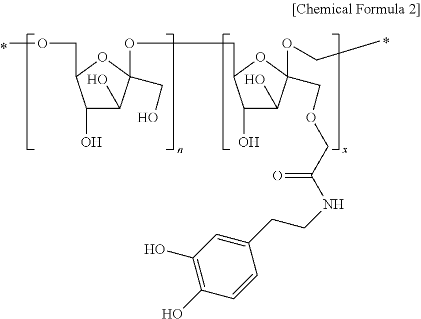

In Chemical Formula 2,

The n and x are mole fractions within the repeating unit, respectively, in which 0.7≤n≤0.95, 0.05≤m≤0.3, and n+x=1.

The weight ratio of the carboxymethylated levan and dopamine may be 1:0.5 to 0.9.

Further, another aspect of the present disclosure provides a tissue adhesion composition including a levan-catechol composite according to the present disclosure.

The tissue adhesion composition may further include ferric chloride, sodium periodate, horseradish peroxidase and hydrogen peroxide, fibrinogen or a mixture thereof as a cross-linking agent.

The tissue adhesion composition may be used for hemostasis, wound healing or closure.

Further, yet another aspect of the present disclosure provides a method for preparing a levan-catechol composite including (a) carboxymethylating levan to obtain carboxymethylated levan; and (b) conjugating the carboxymethylated levan to dopamine through EDC-NHS coupling.

Further, still another aspect of the present disclosure provides a nanocluster including a levan-catechol composite according to the present disclosure; and a hydrophobic material.

The nanocluster may have a core-shell form including a core containing the hydrophobic material; and a shell surrounding the core and containing the levan-catechol composite.

The average diameter of the nanocluster may be 1 to 600 nm.

The hydrophobic material may include magnetic nanoparticles, metal nanoparticles, drugs, or a combination thereof.

The magnetic nanoparticles may be at least one selected from iron (II) oxide, iron (III) oxide, cobalt ferrite, zinc ferrite, nickel ferrite, manganese ferrite, iron, cobalt, nickel, manganese, FeAu, FePt and CoNi; and the metal nanoparticles may be at least one selected from gold, silica, titania and magnesium. The drugs may be at least one material selected from the group consisting of hydrophobic drugs, nucleic acids, proteins, polypeptides, carbohydrates, inorganic materials, antibiotics, anticancer agents, antimicrobial agents, steroids, anti-inflammatory analgesics, sex hormones, immunosuppressants, antiviral agents, anesthetics, antiemetics, antihistamines, local anesthetics, antiangiogenic agents, vasoactive agents, anticoagulants, immunomodulators, cytotoxic agents, antibodies, neurotransmitters, psychotropic drugs, oligonucleotides, lipids, cells, tissues, cancer chemotherapeutic agents and vaccines.

Still another aspect of the present disclosure provides a tissue adhesion composition including a nanocluster according to the present disclosure.

The nanocluster included in the tissue adhesion composition may include iron oxide nanoparticles as the hydrophobic material.

Still another aspect of the present disclosure provides a drug delivery system including a nanocluster according to the present disclosure.

The nanocluster included in the drug delivery system may be a hydrophobic material, and may include a magnetic nanoparticle-drug composite formed by conjugating the magnetic nanoparticles and the drug.

Still another aspect of the present disclosure provides a method for preparing a nanocluster, including (A) electrospraying a solution for preparing a nanocluster, in which a levan-catechol composite according to the present disclosure and a hydrophobic material are dispersed, into an aqueous solution.

The solution for preparing the nanocluster may be obtained by mixing a first dispersion solution in which the levan-catechol composite is dispersed in a first solvent; and a second dispersion solution in which the hydrophobic material is dispersed in a second solvent; and the first solvent and the second solvent may be the same as or different from each other, and may each independently be at least one selected from the group consisting of hexane, toluene, tetrahydrofuran (THF), dimethylformamide (DMF), dimethyl sulfoxide (DMSO), and alcohol-based compounds.

Still another aspect of the present disclosure provides a tissue adhesion method including treating a tissue adhesion composition including a levan-catechol composite according to the present disclosure, or a tissue adhesion composition including a nanocluster according to the present disclosure, to a subject in need thereof.

### Advantageous Effects

According to the present disclosure, it is possible to be applied for use in tissue adhesion in wet environments, wound healing, hemostasis, or drug delivery by preparing a levan-catechol composite by the conjugation of levan and catechol, and by hydrogelating or nanoclustering the same.

Meanwhile, effects to be achieved in the present disclosure are not limited to the aforementioned effects, and other not-mentioned effects will be obviously understood by those skilled in the art from the description below.

### DESCRIPTION OF DRAWINGS

FIG. 1 shows (a) a schematic diagram of a synthetic process of a levan-catechol composite (LC), (b) FTIR spectra of levan, carboxymethylated levan (CM-L), and LC, (c)¹H NMR spectra of levan (top), DOPA (middle), and LC (bottom) in D₂O, and (d) UV-VIS spectra of levan and LC.

FIG. 2 shows swelling behavior and morphological analysis results of LC hydrogels: (a) swelling behavior of hydrogels at 37° C. (n=3) and 1×PBS conditions, (b) photographic images of the hydrogels before and after swelling, (c) SEM images of dried hydrogels LC-Fe³⁺ and LC-IO₄⁻, (d) a graph showing average pore areas, (e) a graph showing hydrogel porosity (%), and (f) images showing crosslinking states of LC hydrogels according to a crosslinking agent.

FIG. 3 shows graphs showing results of immunogenicity analysis of the LC hydrogels, including the levels of (a) TNF-α and (b) IL-6 cytokines released from Raw 264.7 cells after exposure to 0.5 μg/mL LPS and levan and LC at different concentrations (n=6).

FIG. 4 shows results of evaluating the tissue adhesion of LC hydrogels: (a) a schematic diagram of an in-vitro lap shear experiment, (b) an image of a lap shear experiment using a porcine skin tissue surface, (c) a strain-stress curve graph obtained through the lap shear-stress experiment, (d) a maximum lap shear-stress graph, (e) a Young's modulus graph, and (f) an adhesion energy graph (n=5).

FIG. 5 shows results of cell viability and cell migration analysis of LC hydrogels: (a) cell viability (NIH3T3 cells and HaCaT cells) after hydrogel extract treatment, (b) optical microscopy images of cell migration after hydrogel extract treatment, and (c) graphs corresponding to scratch area closures.

FIG. 6 shows In vitro hemostatic efficacy evaluation of LC hydrogels: (a) an image of hemolysis and (c) an image of a coagulation state, (b) a graph of hemolysis rates (%) (n=3), (d) a graph of BCIs at various time points, and (e) SEM images of platelet adhesion in (i) LC-Fe³⁺ and (ii) LC-IO₄⁻; and in vivo hemostatic efficacy evaluation, which shows (f) an experimental image confirming a hemostasis level in a hemostatic model using rat liver, and (g) a graph of an experimental result of comparing amounts of blood eluted when a hemostatic agent was treated in rat liver.

FIG. 7 shows results of evaluating the wound healing efficacy of LC hydrogels: (a) images of skin incisions treated with fibrin glue (FG) and hydrogel samples (LC-Fe³⁺ and LC-IO₄⁻) and a nontreated group (NT), and (b) a graph showing a wound closure rate % (n=5) of skin incisions treated with the nontreated group (NT), fibrin glue (FG), LC-Fe³⁺, and LC-IO₄⁻.

FIG. 8 shows results of evaluating the wound healing efficacy of the LC hydrogels: (a) images of hematoxylin and eosin (H&E) staining results 3, 7, and 14 days after incision (wound edges are indicated with black arrows), (b) Masson's trichrome (MT) staining images, (c) a graph showing wound surface width, (d) a graph showing epithelial tongue length, and (e) a graph showing the number of neutrophils (n=3) in the wounds 7 days after incision.

FIG. 9 is a schematic diagram showing (a) a process for preparing a levan-catechol-iron oxide nanocluster (LC-IO) through electrospraying of a levan-catechol composite and iron oxide (magnetic nanoparticles) according to the present disclosure, and (b) a process for preparing a levan-catechol-iron oxide-doxorubicin nanocluster (LC-IO-Dox) through electrospraying of a levan-catechol composite, iron oxide, and doxorubicin (drug) according to the present disclosure.

FIG. 10 shows (a) TEM images of a levan-catechol-iron oxide nanoclusters (LC-IO) and a levan-catechol-iron oxide-doxorubicin nanocluster (LC-IO-Dox) according to the present disclosure, and (b) a graph showing a diameter distribution of levan-catechol-iron oxide nanoclusters (LC-IO).

FIG. 11 is (a) a schematic diagram showing a process for preparing a levan-catechol-iron oxide-doxorubicin nanocluster (LC-IO-Dox) in a core-shell form through electrospraying of a levan-catechol composite, iron oxide, and doxorubicin (drug) according to the present disclosure and a schematic diagram showing active targeting of breast cancer through GLUT receptors by the prepared LC-IO-Dox, and (b) a schematic diagram showing a process in which the levan-catechol-iron oxide-doxorubicin nanocluster (LC-IO-Dox) is introduced into a cell and then decomposed by enzymes present in the body to release Dox as an anticancer agent.

FIG. 12 shows (a) a graph of fluorescence emission intensity of Dox according to a concentration of doxorubicin (Dox) (Dox standard curve) and (a) a graph of fluorescence emission intensity of Dox over time in a levan-catechol-iron oxide-doxorubicin nanocluster (LC-IO-Dox).

FIG. 13 shows graphs of evaluating the antimicrobial activities of (a) levan, (b) carboxymethylated levan, and (c) a levan-catechol composite, and (d) an image of E. coli culture media according to a treatment material.

FIG. 14 is a graph evaluating antimicrobial activity of levan-catechol-iron oxide nanoclusters (LC-IO).

FIG. 15 shows results of evaluating the tissue adhesion of LC-IO nanoclusters: (a) a schematic diagram of an in-vitro lap shear experiment, (b) a strain-stress curve graph obtained through a lap shear-stress experiment, (c) a maximum lap shear-stress graph according to a concentration of LC-IO, (d) a Young's modulus graph according to a concentration of LC-IO, and (f) an adhesion energy graph according to a concentration of LC-IO (n=5).

FIG. 16 shows results of evaluating In vitro hemostatic efficacy of LC-IO nanoclusters: (a) an image showing a hemolysis state and (b) a graph showing a hemolysis rate (%) (n=3).

FIG. 17 shows results of cell viability and cell migration analysis of LC-IO nanoclusters: (a) graphs showing cell viabilities (L929 cells and HaCaT cells) after LC-IO nanocluster treatment, (b) graphs showing a scratch area closure, and (c) an optical microscopy image of cell migration after nanocluster treatment.

FIG. 18 shows results of evaluating the wound healing efficacy of LC-IO nanoclusters: (a) a schematic diagram showing a skin incision experiment in rats, (b) images of skin incisions after treatment with fibrin glue (FG) and LC-IO nanoclusters and in a nontreated group (NT), and (c) a graph showing a wound closure rate % of skin incisions treated with the nontreated group (NT), fibrin glue (FG), and LC-IO nanoclusters (n=5).

FIG. 19 shows results of evaluating the wound healing efficacy of LC-IO nanoclusters: (a) images of hematoxylin and eosin (H&E) staining results 3, 7, and 14 days after incision (wound edges are indicated by black arrows), (b) Masson's trichrome (MT) staining images, (c) a graph showing wound surface width, and (d) a graph showing epithelial tongue length.

FIG. 20 is a hematoxylin and eosin (H&E) staining image for a first skin adhesion experiment, as a result of evaluating the wound healing efficacy of LC-IO nanoclusters.

### MODES

Hereinafter, exemplary embodiments of the present disclosure will be described in detail with reference to the accompanying drawings. The exemplary embodiments of the present invention may be modified in various forms, and it should not be construed that the scope of the present invention is limited to exemplary embodiments to be described below. The exemplary embodiments will be provided for more completely explaining the present invention to those skilled in the art. Therefore, the shapes of components in the drawings are exaggerated to emphasize a clearer explanation.

Unless otherwise defined, all terms (including technical and scientific terms) used in the present specification may be used as the meaning which may be commonly understood by the person with ordinary skill in the art, to which the present disclosure pertains. Terms defined in commonly used dictionaries should not be interpreted in an idealized or excessive sense unless expressly and specifically defined.

Hereinafter, a levan-catechol composite according to the present disclosure, a method for preparing the same and uses thereof; and a nanocluster including the levan-catechol composite, a method for preparing the same and uses thereof will be described in detail.

### Levan-Catechol Composite

According to the present disclosure, there is provided a levan-catechol composite including carboxymethylated levan and dopamine.

The levan is a natural material first discovered by Cooper in 1935 and is found in some plants such as barley, cactus, and grasses including onions and garlic, and microorganisms such as Bacillus. As a specific example, the levan may be produced by gram-negative bacteria such as Zymomonas mobilis, Erwinia amylovora, and Halomonas spice AAD6. The levan is a type of fructan in which monomers are formed through β-(2,6) single bonds, and unlike inulin, which is a fructan mainly having a β-(2,1) linked structure, the levan is present in both β-(1,6) and β-(2,6). The inulin exhibits a low molecular weight and insolubility, whereas the levan is composed of a relatively high molecular weight and is a natural material that has a characteristic of very high water solubility, and has the functions of promoting mineral absorption and promoting the growth of intestinal lactic acid bacteria, and thus has been developed for various uses, such as pharmaceuticals and food additives.

In the present disclosure, carboxymethylation means a reaction in which a hydrogen atom bonded to carbon, nitrogen, oxygen, sulfur atom, etc. of an organic compound is replaced with a carboxymethyl group (—CH₂—COOH). As a specific example, the carboxymethylated levan may mean levan containing a carboxymethyl group as a substituent by replacing a hydrogen atom contained in levan with a carboxymethyl group.

The carboxymethylated levan may include a repeating unit represented by the following Chemical Formula 1.

Endotoxin, as LPS released by most gram-negative bacteria, is a pyrogen that induces a strong innate immune response, and the presence of endotoxin in the blood may lead to hypotension, respiratory failure, and reduced oxygen delivery. Accordingly, removal of endotoxins (contamination during microbial production) is required for biomedical applications. In the present disclosure, as shown in Chemical Formula 1 below, the hydrogen atoms present in the repeating unit of levan are replaced with carboxymethyl groups, thereby significantly reducing the endotoxin of levan.

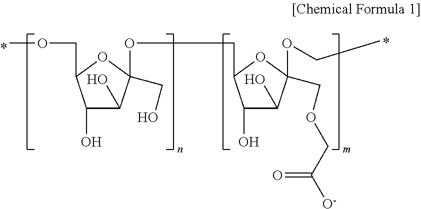

In Chemical Formula 1,
the n and m are mole fractions within the repeating unit, respectively, in which 0.7≤n≤0.95, 0.05≤m≤0.3, and n+m=1.

The levan-catechol composite may include a repeating unit represented by the following Chemical Formula 2. As described above, the levan-catechol composite including the repeating unit of the following Chemical Formula 2 may be formed through the conjugation of carboxymethylated levan of Chemical Formula 1 with significantly reduced endotoxin and dopamine, and the levan-catechol composite formed above may have the characteristic of biocompatibility due to significantly reduced toxicity.

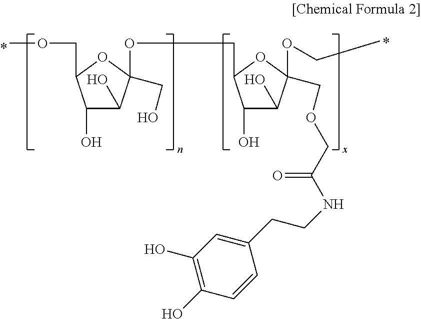

In Chemical Formula 2,

The n and x are mole fractions within the repeating unit, respectively, in which 0.7≤n≤0.95, 0.05≤m≤0.3, and n+x=1.

In Chemical Formula of the present disclosure, “*” means a moiety connected to the same or different atom or Chemical Formula.

The weight ratio of the carboxymethylated levan and dopamine may be 1:0.5 to 0.9, 1:0.6 to 0.8 or 1:0.7 to 0.8. Within the weight ratio range, in the formation of the levan-catechol composite, the conjugation of carboxymethylated levan and dopamine may be facilitated, and the levan-catechol composite thus formed has an effect of improving the adhesive ability to skin tissue.

According to an exemplary embodiment of the present disclosure, there is provided a tissue adhesion composition including the levan-catechol composite according to the present disclosure. Conventionally, levan had the disadvantages of having low intrinsic viscosity and being diluted and washed away in wet environments, but according to the present disclosure, a levan-catechol composite in which carboxymethylated levan and dopamine are conjugated has excellent tissue adhesion even in wet environments. Accordingly, the levan-catechol composite may be applied as a tissue adhesion composition.

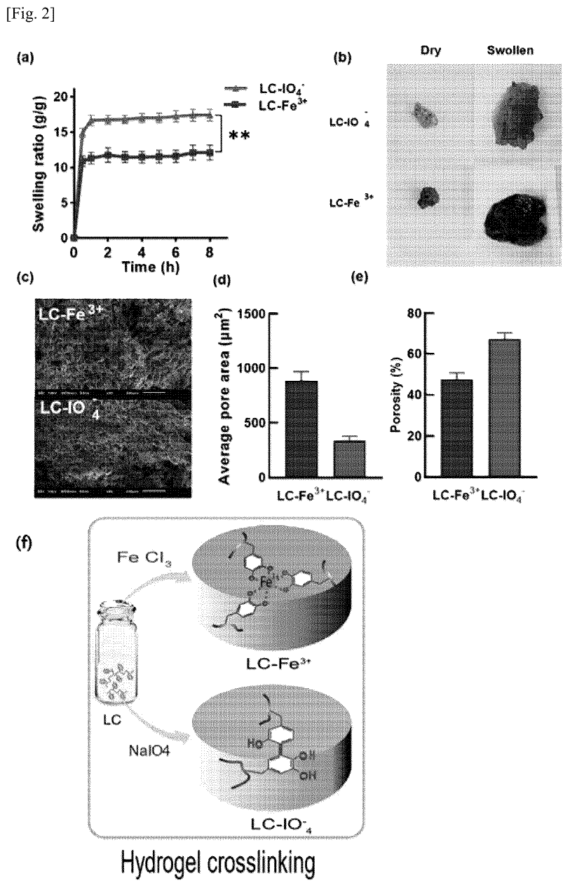

The composition may further include iron chloride (FeCl₃), sodium periodate (NaIO₄), horseradish peroxidase and hydrogen peroxide (HRP/H₂O₂), fibrinogen or a mixture thereof as a cross-linking agent. When the tissue adhesion composition according to the present disclosure further includes the cross-linking agent described above, a plurality of levan-catechol composites may be coordinately bonded or covalently bonded to each other to form hydrogels. When the composites are coordinated to each other, the coordination may be mediated by metals or metal ions. That is, the tissue adhesion composition may be in the form of a hydrogel. Referring to FIG. 2F, the bonding structure of the hydrogel formed depending on a type of cross-linking agent may be confirmed.

The tissue adhesion composition may be used for hemostasis, wound healing or closure. Since the levan-catechol composite has excellent hemostatic effect, wound healing promoting effect, and tissue adhesion effect, the tissue adhesion composition containing the levan-catechol composite may be applied for used of hemostasis, wound healing, and skin or tissue closure. As a specific example, the tissue adhesion composition may be applied to any one selected from the group consisting of, but not limited to, neurosurgery including vascular surgery areas, orthopedic surgery including adhesion of bones, hemostasis in patients with thermal injuries, closure of the femoral artery, cataract incision closure, cartilage healing, skin conjugation, hemostasis of incision surfaces of organs/glandules, gastrointestinal syndesmosis, and tendon/ligament healing.

Further, the present disclosure provides a tissue adhesion method including treating a tissue adhesion composition including the levan-catechol composite according to the present disclosure, or a tissue adhesion composition including the nanocluster according to the present disclosure, to a subject in need thereof.

In the present disclosure, the term “subject” refers to a subject in need of hemostasis, and more particularly, may mean mammals such as humans or non-human primates, mice, dogs, cats, horses and cattle, but is not limited thereto.

According to an exemplary embodiment of the present disclosure, there is provided a method for preparing a levan-catechol composite, including (a) carboxymethylating levan to obtain carboxymethylated levan; and (b) conjugating the carboxymethylated levan to dopamine through EDC-NHS coupling.

The step (a) may be performed through a process of mixing levan, sodium hydroxide, and chloroacetic acid. At this time, the levan and chloroacetic acid may be mixed in a weight ratio of 1:0.7 to 1, 1:0.8 to 1, or 1:0.9 to 1. Within the weight ratio range, there is an effect of significantly reducing the endotoxin of levan. Accordingly, the toxicity of the formed levan-catechol composite is reduced to have an advantage of excellent biocompatibility.

The levan used as an initial raw material may be obtained through cell disruption of microorganisms using a mechanical or physical method, or may be obtained through cell disruption of microorganisms using a non-mechanical or chemical method.

The step (b) may be performed by carbodiimide-promoted conjugation between a carboxyl group introduced into carboxymethylated levan and an amine group of dopamine, and specifically, may be performed through a process of reacting a reaction mixture including carboxymethylated levan, dopamine, N-hydroxysuccinicimide (NHS), and 1-ethyl-3-(3-dimethylaminopropyl)carbodiimide (EDC). More specifically, the process of reacting the reaction mixture may be performed by adjusting the reaction mixture to pH 4 to 6 or pH 5, mixing the reaction mixture at 400 to 600 rpm for 5 to 30 hours, 5 to 24 hours, or 5 to 12 hours under room temperature conditions under argon, dialyzing the reaction mixture against acidic distilled water having pH 5 to 6 or pH 5.5 at 1 to 10° C., 2 to 6° C., or 3 to 5° C. for 1 to 3 days or 2 days, dialyzing the mixture against deionized water for 1 to 10 hours or 3 to 5 hours, and then freeze-drying.

At this time, carboxylated levan, dopamine, NHS and EDC may be mixed in a weight ratio of 1:0.4 to 0.7:0.8 to 1:0.5 to 0.9, or 1:0.5 to 0.6:0.9 to 1:0.7 to 0.8. Within the weight ratio range, in the formation of the levan-catechol composite, the conjugation of carboxymethylated levan and dopamine may be facilitated, and the levan-catechol composite thus formed has an effect of improving the adhesive ability to skin tissue.

### Nanocluster Including Levan-Catechol Composite

According to the present disclosure, there is provided a nanocluster including a levan-catechol composite according to the present disclosure and a hydrophobic material. Although there have been cases where nanofibers or nanoparticles were manufactured using conventional electrospraying techniques, the nanofibers or nanoparticles had the disadvantage of easily dissolving and swelling, resulting in low stability and only functioning as simple carriers. On the other hand, in the present disclosure, through electrospraying of a mixed solution of a levan-catechol composite containing levan, which is a natural polysaccharide with amphiphilic properties and a bioactive material function, and a hydrophobic material, there were prepared nanoclusters that were not easy to dissolve/rupture or swell in an aqueous environment, but are also easily recovered and promote tissue adhesion in an aqueous environment, accelerate a wound healing rate, and are capable of releasing therapeutic drugs.

The nanocluster may have a core-shell form including a core containing the hydrophobic material; and a shell surrounding the core and containing the levan-catechol composite. During the electrospraying process of the mixed solution of the levan-catechol composite and the hydrophobic material, a stable core-shell type nanocluster may be formed through self-assembly by hydrophobic interactions and electrostatic interactions. The nanocluster formed at this time may maintain multifunctional properties such as physiological activity and adhesive performance due to the shell containing the levan-catechol composite, and in particular, have the characteristic of biocompatibility by including the levan-catechol composite with significantly reduced toxicity due to the reduction of endotoxin.

The average diameter of the nanoclusters may be 1 to 600 nm, 50 to 300 nm, 100 to 200 nm or 140 to 150 nm. Within the average diameter range, there are effects of excellent tissue adhesion, wound healing promotion ability, hemostatic efficacy, and drug release ability.

The hydrophobic material may include magnetic nanoparticles, metal nanoparticles, drugs, or a combination thereof, and the magnetic nanoparticles may be at least one selected from iron (II) oxide, iron (III) oxide, cobalt ferrite, zinc ferrite, nickel ferrite, manganese ferrite, iron, cobalt, nickel, manganese, FeAu, FePt and CoNi; and the metal nanoparticles may be at least one selected from gold, silica, titania and magnesium. The drugs may be at least one material selected from the group consisting of hydrophobic drugs, nucleic acids, proteins, polypeptides, carbohydrates, inorganic materials, antibiotics, anticancer agents, antimicrobial agents, steroids, anti-inflammatory analgesics, sex hormones, immunosuppressants, antiviral agents, anesthetics, antiemetics, antihistamines, local anesthetics, antiangiogenic agents, vasoactive agents, anticoagulants, immunomodulators, cytotoxic agents, antibodies, neurotransmitters, psychotropic drugs, oligonucleotides, lipids, cells, tissues, cancer chemotherapeutic agents and vaccines. The drugs are not limited to the types, and may include any drugs that may be delivered into the human body by a carrier.

According to an exemplary embodiment of the present disclosure, the drug may be a hydrophobic drug. At this time, the hydrophobic drug may be at least one selected from the group consisting of doxorubicin, paclitaxel, methotrexate, 5-fluorouracil, mitomycin-C, styrene maleic acid neocarzinostatin (SMANCS), cisplatin, carboplatin, carmustine (BCNU), dacarbazine, etoposide, and daunomycin, but is not limited thereto.

According to an exemplary embodiment of the present disclosure, the nucleic acid may be DNA or RNA. At this time, the RNA may be siRNA, miRNA or mRNA, but is not limited thereto.

According to an exemplary embodiment of the present disclosure, there is provided a tissue adhesion composition including the nanocluster according to the present disclosure.

The nanocluster included in the tissue adhesion composition may include iron oxide nanoparticles as the hydrophobic material, and in this case, there are effects of exhibiting excellent tissue adhesion, wound healing promotion ability, and hemostatic efficacy. In addition, the tissue adhesion composition has the characteristic of biocompatibility by including the levan-catechol composite with significantly reduced toxicity due to a decrease in endotoxin.

The tissue adhesion composition including the nanocluster may be used for hemostasis, wound healing or closure. Since the levan-catechol composite included in the nanocluster has excellent hemostatic effect, wound healing promoting effect, and tissue adhesion effect, the tissue adhesion composition containing the levan-catechol composite may be applied for used of hemostasis, wound healing, and skin or tissue closure. As a specific example, the tissue adhesion composition may be applied to any one selected from the group consisting of, but not limited to, neurosurgery including vascular surgery areas, orthopedic surgery including adhesion of bones, hemostasis in patients with thermal injuries, closure of the femoral artery, cataract incision closure, cartilage healing, skin conjugation, hemostasis of incision surfaces of organs/glandules, gastrointestinal syndesmosis, and tendon/ligament healing.

According to an exemplary embodiment of the present disclosure, there is provided a drug delivery system including the nanocluster according to the present disclosure. The drug delivery system has the characteristic of biocompatibility by including the levan-catechol composite with significantly reduced toxicity due to a decrease in endotoxin.

The nanocluster included in the drug delivery system may be a hydrophobic material, and may include a magnetic nanoparticle-drug composite formed by conjugating the magnetic nanoparticles and the drug. Specifically, the drug composite may be an iron oxide-hydrophobic drug composite formed by conjugating iron oxide nanoparticles and a hydrophobic drug, and in this case, there is an effect of exhibiting excellent drug release efficacy.

According to an exemplary embodiment of the present disclosure, there is provided a method for preparing a nanocluster, including (A) electrospraying a solution for preparing a nanocluster, in which a levan-catechol composite according to the present disclosure and a hydrophobic material are dispersed, into an aqueous solution.

Specifically, the electrospraying process of step (A) may be performed by electrospraying in a water tank containing an aqueous solution under magnetic stirring, by using 20 to 25 gauge or 22 to 24 gauge needles and applying a voltage of 20 to 25 kV or 22 to 24 kV, a flow rate of 2 to 10 or 4 to 6 μL/min, and a nozzle distance of 10 to 15 cm or 11 to 13 cm. At this time, the aqueous solution contained in the water tank may be distilled water, but is not limited thereto.

In addition, after step (A), the method may further include (B) collecting the nanoclusters generated simultaneously with the electrospraying using a magnet. Specifically, the magnet may be a neodymium magnet. The nanoclusters collected as described above may be redispersed in 10 to 20% or 14 to 15% DMSO for long-term storage.

The solution for preparing the nanocluster may be obtained by mixing a first dispersion solution in which the levan-catechol composite is dispersed in a first solvent; and a second dispersion solution in which the hydrophobic material is dispersed in a second solvent. The first solvent and the second solvent may be the same as or different from each other, and may each independently be at least one selected from the group consisting of hexane, toluene, tetrahydrofuran (THF), dimethylformamide (DMF), dimethyl sulfoxide (DMSO), and alcohol-based compounds. Specifically, the first solvent may be DMSO, and the second solvent may be hexane, but are not limited thereto.

In step (A), the size of the hydrophobic material dispersed in the solution for preparing the nanocluster may be 1 to 20 nm, 5 to 15 nm, or 7 to 12 nm. Within the size range, self-assembly is easy during electrospraying of the solution for preparing the nanocluster, thereby preparing stable core-shell type nanoclusters.

According to an exemplary embodiment of the present disclosure, the hydrophobic material of step (A) may include a magnetic nanoparticle-drug composite formed by conjugating magnetic nanoparticles and a drug. Specifically, the drug composite may be an iron oxide-hydrophobic drug composite formed by conjugating iron oxide nanoparticles and a hydrophobic drug, and in this case, there is an effect of exhibiting excellent drug release efficacy.

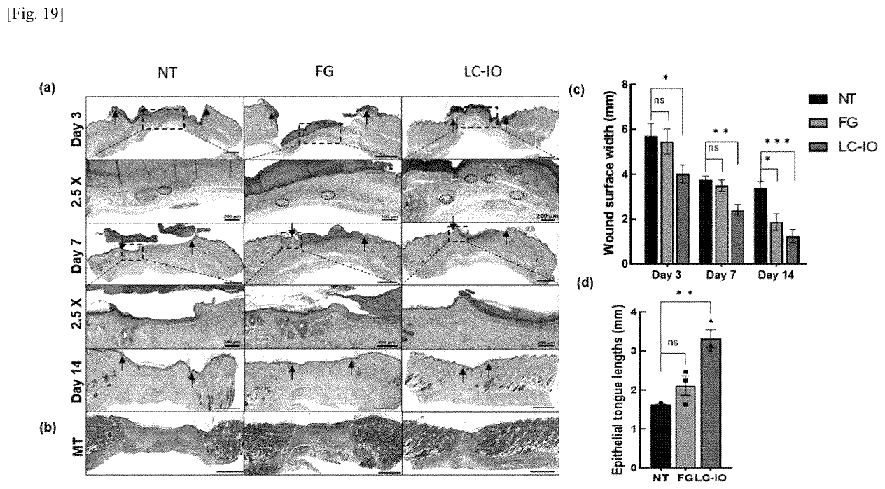

As such, when the hydrophobic material is the magnetic nanoparticle-hydrophobic drug composite, the nanocluster finally generated by electrospraying may have a core-shell form including a core containing the magnetic nanoparticles; a shell surrounding the core and containing the levan-catechol composite; and a hydrophobic drug dispersed in the core and the shell (see FIG. 19).

Breast cancer is the most common cancer in women and consumes high-energy that causes overexpression of a Glut 5 receptor, called a Warburg effect. Doxorubicin (Dox), an anthracycline-based antibiotic, has anticancer effects by inhibiting topoisomerase II (topo II), a key enzyme in DNA replication, but Dox-based chemotherapy has the disadvantage of not specifically targeting breast cancer cells. Referring to FIG. 19, the levan-catechol-iron oxide nanocluster (LC-IO) according to the present disclosure may be utilized for active targeting of breast cancer through a GLUT 5 receptor by loading doxorubicin in LC-IO to form a levan-catechol-iron oxide-doxorubicin nanocluster (LC-IO-Dox) (see A of FIG. 11). In this case, the concentration of doxorubicin in the LC-IO-Dox may be 0.1 to 10 μg/ml, 0.5 to 5 μg/ml or 0.5 to 1.5 μg/ml, but is not limited thereto.

The magnetic nanoparticle-drug composite may be obtained by adding the magnetic nanoparticles and the drug to a third solvent and then performing sonication. The third solvent is the same as or different from the first solvent or the second solvent, and may be at least one selected from the group consisting of hexane, toluene, tetrahydrofuran (THF), dimethylformamide (DMF), dimethyl sulfoxide (DMSO), and alcohol-based compounds. Specifically, the third solvent may be hexane, but is not limited thereto.

The above description just illustrates the technical spirit of the present disclosure and various changes and modifications may be made by those skilled in the art to which the present disclosure pertains without departing from an essential characteristic of the present disclosure. Accordingly, the various exemplary embodiments disclosed in the present disclosure are not intended to limit the technical spirit but describe the present disclosure and the technical spirit of the present disclosure is not limited by the following exemplary embodiments. The protective scope of the present disclosure should be construed based on the following claims, and all the techniques in the equivalent scope thereof should be construed as falling within the scope of the present disclosure.

Hereinafter, the present invention will be described in more detail through Examples.

### <Materials>

Levan produced by Zymomonas mobilis (MW=95 kDa, DP=587) was purchased from Real biotech (Gongiu, Korea), chloroacetic acid and sodium periodate were purchased from Junsi Chemical (Tokyo, Japan), 1-ethyl-3-(3-dimethylaminopropyl) carbodiimide (EDC) was purchased from APExBIO (Boston, USA), dopamine, N-hydroxysuccinimide (NHS), iron (III) chloride, lipopolysaccharides from Escherichia coli o55:b5 (LPS), and penicillin streptomycin were purchased from Sigma-Aldrich (St. Louis, MO, USA). In addition, hydrochloric acid, sodium hydroxide, isopropanol, and ethanol were purchased from Samchun (Seoul, Korea). In addition, PBS (10×) was purchased from LPS solution (Daejeon, Korea) and diluted to a final concentration of 137 mM NaCl, 10 mM phosphate, and 2.7 mM KCl (1×PBS). In addition, a Greenplast Q prefilled syringe kit (fibrin glue kit) was purchased from GC Biopharma (Yongin, Korea) and porcine skin was purchased from Stellen Medical (St. Paul, MN, USA). In addition, the cell lines used in the experiments of this study were purchased from the American Type Culture Collection (Manassas, VA, USA). Fetal bovine serum (FBS) was purchased from Gibco (Seoul, Korea), L-glutamine was purchased from Lonza (Walkersville, MD, USA), a high glucose Dulbecco's modified eagle medium (DMEM) and a Dulbecco phosphate buffered saline (DPBS) were purchased from HyClone (Logan, UT, USA), and a DMEM for a Raw 264.7 cell line was purchased from Welgene fresh media (Gyeongsan, Korea). In addition, the Pierce Chromogenic Endotoxin quanti kit was purchased from Thermo Scientific (Rockford, IL, USA), Culture-Insert 2 wells were purchased from Ibidi (Grafelfing, Germany), and Cell Counting Kit-8 was purchased from MedChemExpress (NJ, USA). In addition, mouse TNE-α and mouse IL-6 DuoSet ELISA kits were purchased from R&D Systems (Minnesota, USA) and blood was collected from rabbits based on the animal experimentation ethics regulations of the Institutional Review Board of POSTECH (IRB number POSTECH-2022-0004).

### EXAMPLES

### Preparation Example 1: Preparation of Carboxymethylated Levan and Removal of Endotoxin

The intrinsic viscosity of levan of Zymomonas mobilis was determined to be 23.4 mL/g, and the molecular weight was determined to be ˜95 kDa using the Mark-Houwink-Sakurada relationship. A levan-catechol composite was synthesized by first preparing carboxymethylated levan (CM-L). Levan (2 g) was dissolved in deionized water (40 mL) and then added with NaOH at a 2:1 (NaOH:levan) molar ratio. The solution was stirred at 500 rpm for 24 hours, and then slowly added with a 1 N NaOH (5 mL) in which chloroacetic acid (1.92 g) was dissolved, over 1 hour, and the solution obtained was stirred at 60° C. for 12 hours. The synthesized CM-L sample was neutralized with dilute NaOH or HCl and then precipitated with isopropanol to be purified. The precipitated CM-L was collected by filtration, dried under vacuum at room temperature, and then characterized by FTIR spectroscopy (Nicolet iS50, USA) and ¹³C NMR (Bruker AV 500 NMR). Levan was incubated with NaOH to activate the hydroxyl groups in a fructan, and conjugated with chloroacetic acid, and the endotoxin was removed from the levan, as described by Govers et al. (Govers, Coen, et al. Bioactive carbohydrates and dietary fibre 8.1 (2016): 15-25.). The endotoxin levels of levan and carboxymethylated levan were determined using a Pierce Chromogenic endotoxin assay kit according to the manufacturer's instructions.

### Example 1: Preparation of Levan-Catechol Composite (LC)

A levan-catechol composite was synthesized by carbodiimide-promoted conjugation between the amine group of dopamine and the introduced carboxyl groups in the CM-L of Preparation Example 1. The CM-L (0.5 g) was dissolved in DI water (100 mL), and then NHS (287.5 mg) and EDC (479.5 mg) were added to the solution to obtain a reaction solution. The reaction solution was stirred for 1 hour, and then added with dopamine (383 mg) to be adjusted to pH 5. The reaction solution adjusted to pH 5 was mixed at 500 rpm at room temperature under argon for 12 hours or more. After the reaction was completed, the reaction solution was dialyzed against acidic distilled water (pH 5.5) for two days and DI water for 4 hours at 4° C., and then freeze-dried to prepare a levan-catechol composite (LC). The reaction success was confirmed by FTIR, NMR, and UV-VIS (Optizen POP Nano Bio, Korea). In addition, the degree of substitution (DS) of the dopamine in the levan-catechol composite (LC) was determined from a ¹H NMR spectrum and Arnow assay.

### Example 2: Preparation of Levan-Catechol Composite (LC) Hydrogel

LC hydrogels were prepared with a 10 wt % levan-catechol composite (LC) solution in phosphate-buffered saline (1 PBS, pH 7.4). NaIO₄ was added to the LC solution at a 2:1 (DOPA:IO₄) molar ratio to covalently couple catechol, and the resulted hydrogel was referred to as “LC-IO₄⁻”. In addition, FeCl₃ was added to the LC solution at a 3:1 (catechol:Fe³⁺) molar ratio and the pH was increased to 9 with 1 N NaOH until the solution turned dark pink, and the resulted hydrogel was referred to as “LC-Fe³⁺”.

### Example 3: Preparation of Levan-Catechol-Iron Oxide Nanocluster (LC-IO) and Levan-Catechol-Iron Oxide-Doxorubicin Nanocluster (LC-IO-Dox)

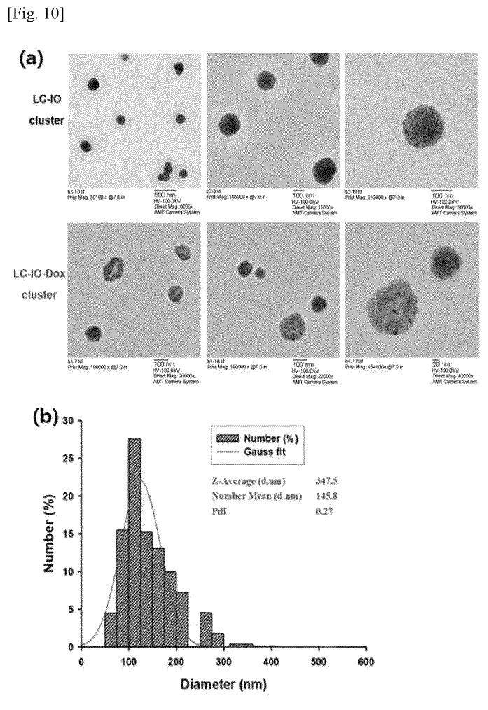

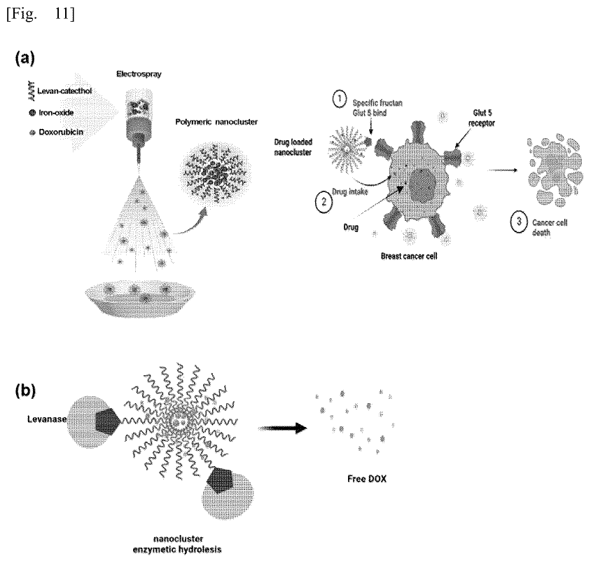

A levan-catechol-iron oxide nanocluster (LC-IO) was prepared using an electrospraying device. The solution for preparing the nanocluster for electrospraying was prepared by adding 1 mL of a hexane solution in which hydrophobic 10 (iron oxide; Fe₃O₄) (Fe 10 mM) nanoparticles having a size of 7 to 12 nm were dispersed to 10 mL of a DMSO solution in which 0.5 wt % LC (the levan-catechol composite of Example 1) was dissolved, and sonicating the solution in a water bath for 1 hour. Next, the prepared solution for preparing the nanocluster was transferred to a syringe and then electrospun into an aluminum bath containing distilled water under magnetic stirring using a 23-gauge needle, a 23 kV voltage, a 5 μL/min flow rate, and a 12 cm nozzle distance. The sprayed nanocluster products were collected using a neodymium magnet and then redispersed in 15% DMSO for long-term storage (see A of FIG. 9). The morphological characteristics of LC-IO may be confirmed through A of FIG. 10, and the average diameter of LC-IO may be confirmed to be 200 nm or less, specifically 145.8 nm through B of FIG. 10.

Meanwhile, the preparation of the levan-catechol-iron oxide-doxorubicin nanocluster (LC-IO-Dox) containing a therapeutic drug was carried out by adding 200 g of Dox to 1 mL of a hexane solution in which IO (iron oxide; Fe₃O₄) (Fe 10 mM) nanoparticles were dispersed and sonicating the solution in a water bath for 1 hour to form an IO-Dox composite, which was prepared in the same manner as the LC-IO preparation process. That is, the LC-IO-Dox was prepared by adding 1 mL of the hexane solution in which LC-IO (100 g) was dispersed to 10 mL of the DMSO solution in which 0.5 wt % LC was dissolved and sonicating the solution in a water bath for 1 hour. Next, the prepared solution for preparing the nanocluster was transferred to a syringe and then electrospun into an aluminum bath containing distilled water under magnetic stirring using a 23-gauge needle, a 23 kV voltage, a 5 μL/min flow rate, and a 12 cm nozzle distance. The sprayed nanocluster products were collected using a neodymium magnet and then redispersed in 15% DMSO for long-term storage (see B of FIG. 9). The morphological characteristics of LC-IO-Dox may be confirmed through A of FIG. 10, and the average diameter of LC-IO-Dox may be confirmed to be 200 nm or less, specifically 145.8 nm through B of FIG. 10.

### Experimental Example

Each experiment was performed at least three times, and the results were presented as mean±standard errors. Statistical significance was determined using a one-tailed t-test, and p<0.05 was considered statistically significant.

### Experimental Example 1. Characterization of Levan-Catechol Composite

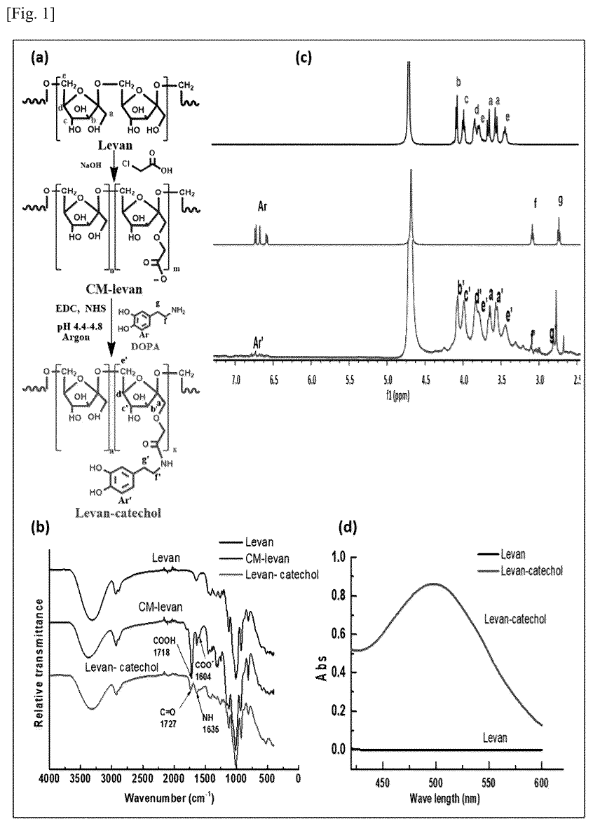

Levan was conjugated to dopamine by first introducing carboxymethyl groups onto a levan chain. The carboxymethyl groups subsequently reacted with amines through EDC chemistry. The successful formation of the composite was confirmed by FTIR spectroscopy, with the spectra of levan and carboxymethylated Levan (CM-L) shown in B of FIG. 1. New bands at about 1718 and 1604 cm⁻¹ in the spectrum of the latter may be assigned to the stretching vibrations of C═O groups in the forms of COOH and COO—, respectively. In addition, a ¹³C NMR spectrum of CM-L exhibits a new peak at 180 ppm that corresponds to carboxylic carbons, which is consistent with the successful formation of CM-L.

EDC chemistry was used to couple the carboxyl group of CM-L with the amino group of dopamine. The synthesized levan-catechol composite (LC) was characterized by FTIR and ¹H NMR spectroscopy and Arnow assay. The FTIR spectrum of LC (B of FIG. 1) represents a carboxyl peak at around 1727 cm⁻¹, which is higher than that of CM-L (˜1718 cm⁻¹) and provides evidence that the sodium salts of the carboxylates (COO⁻) of CM-L had was conjugated to the amino group of DOPA, and the peak at 1635 cm⁻¹ was assigned to amide-group bending.

Dopamine/CM-L conjugation was confirmed by ¹H NMR spectroscopy (C of FIG. 1), which revealed the presence of peaks that corresponded to the aromatic (6.5 to 7 ppm) and methylene protons (3.1 and 2.8 ppm) of DOPA. The degree of substitution (DS) of the catechol groups in LC was calculated from the following equation: DS=A_{aromatic}/(3×A_{4.07}), where A_{aromatic} and A_{4.07} are the integrated areas of the aromatic proton peaks of the catechol moieties and the peak at δ 4.07 (H₆ of levan), respectively, which led to a DS value of approximately 0.07 for LC.

In addition, the Arnow assay was also used to confirm that dopamine had been successfully grafted onto the levan chain. The UV-Vis spectrum of LC (D of FIG. 1) shows a remarkable absorption at 500 nm that is attributable to the catechol groups in LC, while the spectrum of pure levan does not show this peak. The degree of catechol substitution in the levan chain was calculated to be 0.068 from the standard absorbance-concentration curve for dopamine.

### Experimental Example 2. LC Hydrogel Morphology and Swelling Behavior Evaluation

The cross-sectional morphologies of the LC hydrogels were evaluated by scanning electron microscopy (SEM, JSM-6010LV, Jeol, Japan) at 10 kV accelerating voltage. The hydrogels were freeze-dried, cut, and gold-coated for observation.

For the swelling behavior of the hydrogels, freeze-dried LC hydrogel samples were weighed and immersed in 1×PBS (pH 7.4) at 37° C., and three samples were tested per group. At various times, the swelled samples were withdrawn from a buffer and rapidly wiped with filter paper, weighed, and reintroduced to the buffer. This procedure was repeated until the swelling reached a state of equilibrium. The swelling ratio (Q) of each hydrogel was estimated according to the following equation (1):

$$Q = \frac{{W}_{w} - {W}_{d}}{{W}_{d}} \qquad [ Equation 1 ]$$

In which, Ww is the sample weight at each time point and Wd is the dry weight of the sample.

Referring to FIG. 2, both covalently bound LC-IO₄ and non-covalently bound LC-Fe³⁺ exhibited rapid swelling during first 30 minutes of incubation, and swelling equilibrium was observed at 3 hours, and a constant weight was observed for the next 5 hours. For cell growth, biological fluids need to easily penetrate the hydrogels, and it may be confirmed that LC-IO₄⁻ exhibits a significantly higher swelling ratio than LC-Fe³⁺. As may be seen in the SEM image in FIG. 2C, both the hydrogels exhibit relatively uniform porosity. It was confirmed that the pores of LC-IO₄⁻ are slightly smaller than those of LC-Fe³⁺, but have more pores, which may contribute to the high swelling capacity.

### Experimental Example 3. Evaluation of In Vitro Immunogenicities of Levan and Levan-Catechol Composite (LC)

RAW 264.7 mouse macrophage-like cells were grown in fresh Welgene DMEM supplemented with FBS (10 vol %) and penicillin-streptomycin (1 vol %) in a humidified incubator containing 5% CO₂ at 37° C. The cells were seeded on a 96-well plate at a density of 2.0×10⁴ cells per well (300 μL) and maintained in 5% CO₂ for 4 hours at 37° C. After the cells had adhered to the plate, the culture medium (100.0 μL) was replaced with levan or LC solution in DMEM to final concentrations of (1000, 500, 100, or 10 μg/mL), and the cells were incubated with media alone to be used as a negative control (NT). As previously reported, when RAW 264.7 cells were stimulated with lipopolysaccharide (LPS) at 500 ng/mL, the optimal level of TNF-α was released without any cytotoxicity. Therefore, this was selected as a positive control (Nayak, Kaur, & Buttar, 2016). After treatment for 24 hours, each supernatant was collected and stored at −20° C. until further use. The concentrations of IL-6 and TNF-α in the culture supernatants were determined using a DuoSet ELISA kit according to the manufacturer's instructions. The concentrations of IL-6 and TNF-α were determined using standard curves.

Currently, the immunogenicity of levan has not yet been reported. Levan is produced by Zymomonas mobilis, which is a gram-negative bacterium that releases lipopolysaccharides (LPS or endotoxin) derived from the cell membrane. Endotoxin activates the immune system by releasing a variety of pro-inflammatory mediators, such as tumor necrosis factor (TNF-α) and interleukins (IL-1, 6), through monocytes and macrophages. The levan sample was treated under alkaline conditions of 0.62 M NaOH for 24 hours to remove endotoxin, which reduced the level from 1.57 EU/mg for nontreated levan to 0.14 EU/mg for CM-L. The low endotoxin level (0.13 EU/mg) in LC suggests that the LC may be used as a biomedical adhesive.

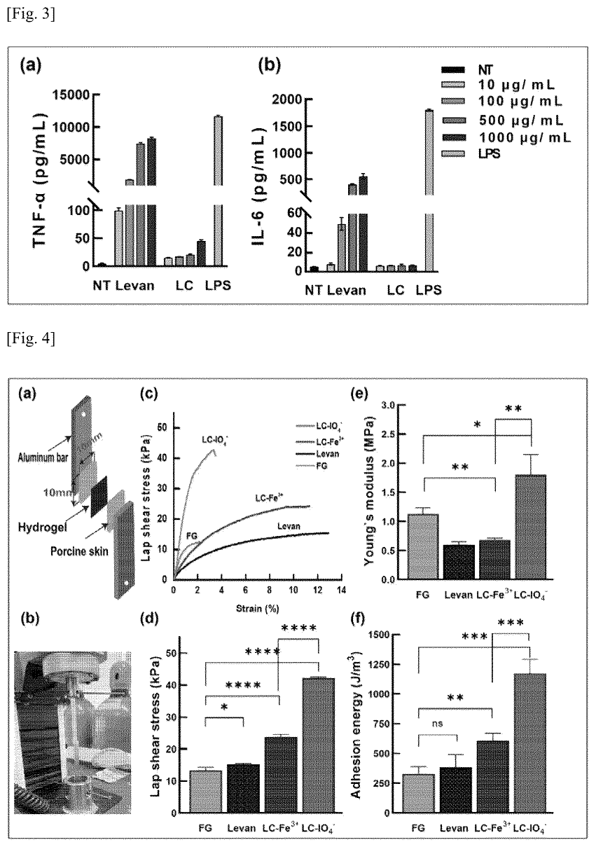

The immunogenicity of levan and LC were evaluated in Raw 264.7 cells, and referring to FIG. 3, the amounts of cytokines released after exposure to the samples for 24 hours were confirmed. Lipopolysaccharide (LPS) was used as a positive control, in which LPS is a major constituent of the cell wall of a gram-negative bacterium, is highly immunogenic, and is one of the best monocyte/macrophage activators. Prior to endotoxin removal, levan exhibited high levels of cytokine production at concentrations of 1 to 0.1 mg/mL and a low response at 10 μg/mL due to its high endotoxin level. The low levels of endotoxin in LC led to a low immune response that was close to that of the negative control. For example, the TNF-α concentration produced by a 1 mg/mL LC sample (44.4±2.4 pg/mL) is sharply lower than that of the levan sample at the same dose (8279.12±134.6 pg/mL) (see FIG. 3). Here, it may be concluded that the induction of inflammatory mediators is endotoxin-dependent and the LC has low immunogenicity and thus excellent biocompatibility.

### Experimental Example 4. Tissue Adhesion Experiment of LC Hydrogel Using Porcine Skin

The adhesion of hydrogels to the biological tissue was evaluated by measuring shear stress using a universal testing machine (Instron 5544, Norwood, MA, US), and the results were shown in FIG. 4. Specifically, porcine skin (stellen Medical, USA) was cut into 10×10 mm pieces, placed in 0.1 M PBS buffer (pH 7.4), and left at 37° C. for 1 hour. The porcine skin that had been left was attached to an aluminum bar having a size of 10×100 mm using an instant adhesive (3M). Then, LC-Fe³⁺ and LC-IO₄⁻ hydrogels and fibrin glue (FG) (GC Biopharma, Korea) as a positive control were applied to the surface of the porcine skin. Then, the aluminum bars attached with the porcine skin without applying the sample were overlapped and the two aluminum bars were fixed using clips. After the two fixed aluminum bars were incubated at 100% relative humidity and room temperature (RT) for 2 hours, the shear stress was measured using a 10 kN load cell at a cross head speed of 10 mm/min until the two aluminum bars were completely separated (see A of FIG. 4).

Referring to FIG. 4, fibrin glue, LC-Fe³⁺ and LC-IO₄⁻ exhibited lap shear stresses of 13.46±0.73 kPa, 24.46±1.2 kPa, and 42.17±0.24 kPa respectively, and LC-IO₄⁻ is significantly at least three times higher than that of commercially available fibrin glues (see C of FIG. 4). Moreover, the adhesions of the two hydrogels are significantly higher than that of a 10 wt % pure levan solution measured under the same wet conditions. In addition, the iron-mediated coordinatively crosslinked hydrogel exhibited adhesion energy that is twice that of the fibrin glue, while the periodate-mediated oxidatively covalently crosslinked hydrogel was found to have four-times higher adhesion energy (see F of FIG. 4).

These results indicate that the LC hydrogels show great adhesion to porcine skin tissue, and thus exhibit a good hemostasis effect. In addition, it is judged that the mechanism of adhesion is due to formation of covalent and non-covalent bonds between accessible nucleophile groups on tissue surfaces such as amino, thiol, and hydroxyl with catechol groups, oxidized o-quinone groups, or hydroxyl groups of levan under wet conditions. That is, in order to increase adhesion to living tissue, a functional group for underwater adhesion, such as catechol, is required to control the disappearance of adhesion in water, and thus, the levan-catechol composite hydrogel of the present disclosure may support strong adhesion even in wet environments.

### Experimental Example 5. Evaluation of Cell Viability and Migration of LC Hydrogel

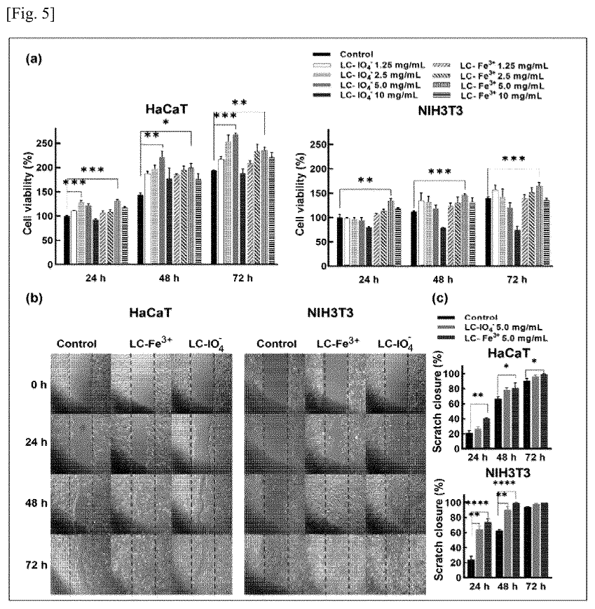

Hydrogel cell viability was evaluated using NIH3T3 and HaCaT cells before an In vitro experiment and then the results were shown in FIG. 5.

Hydrogel cytotoxicity against NIH3T3 and HaCaT cells was evaluated using the CCK-8 assay. The NIH3T3 cells were incubated in DMEM containing 10.0 vol % FBS and 1 vol % penicillin-streptomycin, and the HaCaT cells were incubated in DMEM containing 10.0 vol % FBS, 1 vol % penicillin-streptomycin, and 2 mM L-glutamine in a humidified incubator containing 5% CO₂ at 37° C. Each hydrogel was extracted by soaking the hydrogel sample (1 g) in a supplemented medium (10 mL) at 37° C. for 24 hours. Thereafter, the extract was diluted with the culture medium, and the culture medium itself was used as a control. The NIH3T3 and HaCaT cells were seeded at a density of 5×10³ cells/well in a 96-well plate (200 μL) overnight at 37° C. in 5% CO₂, and 100.0 μL of the medium was replaced with various concentrations of extract dilutions (final adjustment to 10, 5, 2.5, or 1.25 mg/mL). After 24, 48, or 72 hours of incubation, the culture medium was replaced with 100 μL of the culture medium mixed with a Cell Counting Kit-8 solution (10:1 v/v) and further incubated for 1 hour at 37° C. Cell viability was calculated using Equation 2 below.

$$Cell viability = \frac{{OD}_{sample} - {OD}_{blank}}{{OD}_{control} - {OD}_{blank}} \times 100 \qquad [ Equation 2 ]$$

Here, ODₛₐₘₚₗₑ, OD_{control}, and OD_{blank} are the absorbances of tested sample, control (nontreated cells), and blank (a culture medium containing the cck-8 solution) groups at 450 nm measured using a microplate reader (Synergy HTX, USA), respectively.

Meanwhile, in the evaluation of hydrogel cell viability and migration, cell scratching assay was performed using Culture-Insert 2 wells before adding the hydrogel extract to form a cell-free gap with 500±100 m width. After attaching two Culture-Insert wells to a 24-well plate, a cell suspension with a density of 3×10⁵ cells/mL (70 L) was added to each well, and the outer area was filled with a cell culture medium that was cultured for 24 h at 37° C. and 5% CO₂. The two Culture-Insert wells attached with the cells were removed, and non-attached cells were washed with DPBS to be removed, and then added with a hydrogel extract. Gap closure was monitored and imaged at 24-hour intervals by optical microscopy (Optika, Italy).

Referring to FIG. A of 5, both LC-IO₄⁺ and LC-Fe³⁺ were observed to ideally affect NIH3T3 and HaCaT cell growth. The LC-IO₄⁻ and LC-Fe³⁺ promote growth at low levels while inhibiting growth at high levels. Both hydrogels were observed to promote HaCaT cell growth, with viability increasing with increasing concentration in the 1.25 to 5 mg/mL range. The LC-IO₄⁻ extract exhibited higher cell viability than the LC-Fe³⁺ extract. Although cell viability decreased as the concentration was increased to 10 mg/mL, the cell viability was still higher than that of a negative control. The same biphasic pattern was observed for NIH3T3 cells treated with the LC-Fe³⁺ extract, which was found to promote NIH3T3 cell growth more than the LC-IO₄⁻ extract, and observed that viability decreased with increasing concentration. However, even at 10 mg/mL, the LC-IO₄⁻ extract exhibited the cell viability of 70% or higher, which indicates that the LC hydrogels are biocompatible.

Next, the effect of the hydrogel on cell migration was evaluated with 5 mg/mL of the hydrogel extract applied to HaCaT and NIH3T3 cells during wound-scratch testing. B of FIG. 5 shows that the NIH3T3 cells treated with a hydrogel extract migrate significantly faster than those in the control group during co-incubation for 24 hours and 48 hours. The LC-Fe³⁺ and LC-IO₄⁻ extracts and the control group exhibited scratch closures of approximately 74%, 64%, and 24% after 24 hours, respectively. The wound treated with LC-Fe³⁺ was healed after 48 hours, while the control group showed wound regions as high as 62%. On the other hand, only the LC-Fe³⁺ extract elicited significantly different behavior from the control group for 24 hours when applied to HaCaT cells, and 40% of the wound was recovered, but only 21% of the wound was recovered for the control group. NIH3T3 is a murine fibroblast cell line, while HaCaT is an immortalized human keratinocyte cell line. The two types of cells are essential for tissue regeneration following injury.

The good cell-proliferation and fast migration data obtained for the two LC hydrogels are greatly useful for wound healing applications.

### Experimental Example 6. Evaluation of In Vitro and In Vivo Hemostatic Efficacy of LC Hydrogel

### (1) Evaluation of In Vitro Hemostatic Efficacy

To evaluate the hemostatic efficacy of the LC hydrogel, blood coagulation index (BCI), hemolysis analysis, and platelet adhesion experiments were performed.

The blood coagulation index (BCI) of the hydrogel was evaluated as follows. First, a 100 mg sample of each hydrogel was placed in a 5 mL tube and preheated in a 37° C. water bath. 100 μL of a rabbit whole-blood (sodium citrate solution of 3.8%) containing a citrate solution was gently added into the tube to cover the LC hydrogel sample, and then 10 μL of a 0.2 M CaCl₂ solution was added to begin coagulation, while the temperature was maintained at 37° C. Deionized water (2 mL) was carefully added to dissolve unclothed blood while avoiding disturbing the clot at a pre-set time point. The optical density of the supernatant was measured at 540 nm using a microplate reader. The absorbance of citrated whole blood (100 μL) in 2 mL of deionized water was used as a negative control (NC). BCI was calculated using the following Equation 3:

$$BCI = \frac{OD sample}{{OD}_{NC}} \times 100 \qquad [ Equation 3 ]$$

Meanwhile, hemolysis rate and platelet adhesion were evaluated according to the known method (Suneetha, Maduru, Kummara Madhusudana Rao, and Sung Soo Han. ACS omega 4.7 (2019): 12647-12656.). The citrated rabbit whole blood was centrifuged at 4000 rpm for 10 minutes and platelet-rich plasma (PRP) was separated for evaluating platelet adhesion to each hydrogel sample. The red blood cells (RBCs) were washed four times with DBPS and then used for hemolysis assay. To evaluate hydrogel hemocompatibility, 500 μL of diluted RBCs (10 v/v % in DPBS 1×) were added to 100 mg of a hydrogel sample in a microtube and then incubated at 37° C. for 1 hour. In addition, 0.1% Triton-X and DPBS (100 μL) were used as positive and negative controls, respectively. The supernatant was collected after centrifugation at 3500 rpm for 10 minutes and analyzed using a microplate reader at 540 nm. The hemolysis rate was calculated using the following Equation 4 (in triplicate for each group):

$$hemolysis ( \% ) = \frac{( OD sample - OD negative control )}{OD positive control - OD negative control} \times 100 \qquad [ Equation 4 ]$$

According to international standards, a 5% hemolysis value was considered to correspond to no hemolysis in the experiments. For evaluating the platelets adhesion, hydrogels were incubated in a platelet-rich plasma suspension for 1 hour at 37° C. Thereafter, the hydrogels were washed twice with DPBS to remove nonattached platelets and the adhered platelets were incubated and fixed in a 1% glutaraldehyde solution for 2 hours. The hydrogels were then rinsed with DPBS and gradually dehydrated in ethanol solutions and then freeze-dried. The morphologies of the adhered platelets were analyzed by SEM (JSM-6010LV, Jeol, Japan).

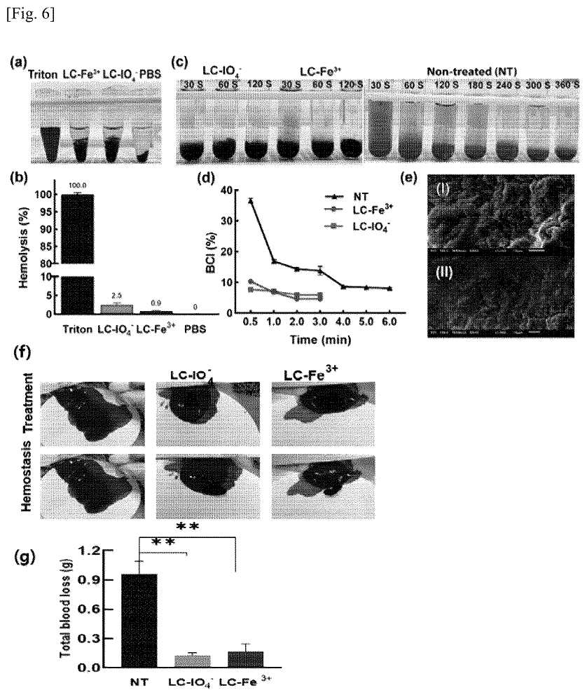

Successful hemostasis promotes subsequent wound healing because coagulation not only prevents blood loss, but the formed fibrin and platelet clot also serve as a temporal lattice required for cells in the inflammatory step, including inflammatory cells and fibroblasts. The In vitro blood coagulation ability of the hydrogels was evaluated using a whole blood-clotting assay, in which a greater blood-clotting index (BCI) indicates slower coagulation. As shown in C and D of FIG. 6, LC-IO₄⁻ exhibited a BCI of 7.65±0.03% after 30 seconds, while the BCI of LC-Fe³⁺ was 10.27±0.4%. In comparison, it was confirmed that the BCI of a blood-only sample (NT) was 36.6±0.53%. This sample needed 6 minutes to achieve the BCI of 8.03±0.21%, which shows that the LC hydrogels significantly increase the rate of blood clotting. A tissue-adhesive material needs to be blood compatible, which is an important safety factor. The hemolysis rate refers to the percentage of red blood cells destroyed by the adhesive material. Low hemolysis corresponds to good hemocompatibility. A of FIG. 6 shows that the hemolysis rate of the LC hydrogels is less than 3%, which is below the limiting value of 5% and confirms that the hydrogels are hemocompatible and are promising materials for hemostasis applications.

Meanwhile, platelet adhesion is a crucial thrombosis indicator because active platelets secrete Polyp that promotes thrombin generation and fibrin clot formation in a bleeding wound. The platelet adhesion was observed by SEM, and the results thereof were shown in E of FIG. 6. The microstructure of the hydrogel plays a role in platelet adhesion in hydrogels with smooth surface microstructures adhere to platelets compared to those with rough surfaces. Both LC-Fe³⁺ and LC-IO₄⁺ exhibited high platelet adhesion that may be caused by rough hydrogel topologies (E of FIG. 6). Through this, it was confirmed that the LC hydrogel clearly accelerated clot formation, thereby exhibiting excellent hemostatic efficacy.

### (2) Evaluation of In Vivo Hemostatic Efficacy

The hemostasis activity was evaluated using a rat liver perforation wound models (Du, Xinchen, et al. Nature communications 12.1 (2021): 4733.). After making an incision in the rat's abdomen, the liver was lifted and laid on top of the pre-weighted filter paper. Thereafter, the liver was incised in 10 mm length to cause bleeding. Then, LC-Fe³⁺ and LC-IO₄⁻ were applied and there was no treatment for a control (NT), and the blood loss was measured (n=3).

F of FIG. 6 shows an image of weighing a hemorrhaged rat soaked in a weighed Whatman paper, and G of FIG. 6 shows a result of analyzing the amount of blood hemorrhaged after adding a hemostatic agent, which shows that the LC hydrogel is more effective in hemostasis than a control group.

### Experimental Example 7. Evaluation of In Vivo Wound Healing Ability of LC Hydrogel

The in vivo wound healing ability of the LC hydrogel was evaluated using a SD rat model. All animal research was carried out in accordance with national regulations and was authorized by Postech's animal experimentation ethical committee (IRB number Postech-2022-0004). To evaluate the bioadhesion and wound-healing properties of the LC hydrogels, Sprague Dawley rats (SD 150 to 200 g, seven-week male) were anesthetized using isoflurane and the backs were shaved. Three 8-mm-diameter circular incisions were made on the back surface of each rat using a biopsy punch. The incisions were quickly sealed by hydrogel (100 mg) or fibrin glue (positive control), and untreated incisions were used as negative controls. The hydrogels were sterilized by washing with 70% ethanol and a DPBS solution (3×). The wounds were photographed on days 0, 1, 3, 5, 7, 10, 14, 17, and 21, and a wound closure rate (%) which was a relative wound closure (%) was calculated using the following Equation 5:

$$Wound closure \% = \frac{{A}_{to} - {A}_{tn}}{{A}_{to}} \times 100 \% \qquad [ Equation 5 ]$$

In which, Aₜₒ and Aₜₙ are wound areas at day 0 and day n (n=1, 3 . . . , 21).

Rats were euthanized on days 3, 7, and 14 post-implantation, the wound size was measured, and then wound sites containing the wound were excised and fixed in a p-formaldehyde solution (3.7 wt %) for subsequent hematoxylin and eosin (H&E) analysis and histological investigation using Masson's trichrome (MT).

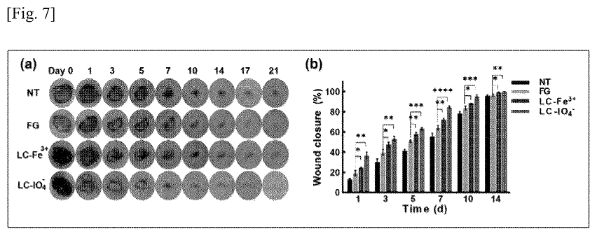

Referring to A of FIG. 7 showing images of skin incisions after treatment with fibrin glue (FG) and hydrogel samples, the wounds treated with the LC hydrogels were closed significantly faster from the first day compared to fibrin-glue-treated wound and untreated wound (nontreated group; NT). In addition, LC-IO₄⁻ had higher healing rate than LC-Fe³⁺, which was determined due to high adhesion of LC-IO₄⁻. The wound treated with LC-IO₄⁻ had recovered by 95.3±1.2% 10 days after incision, while the nontreated wound (NT) and fibrin-glue-treated wound (FG) had healed by 78±1.53% and 83.55±1.7%, respectively.

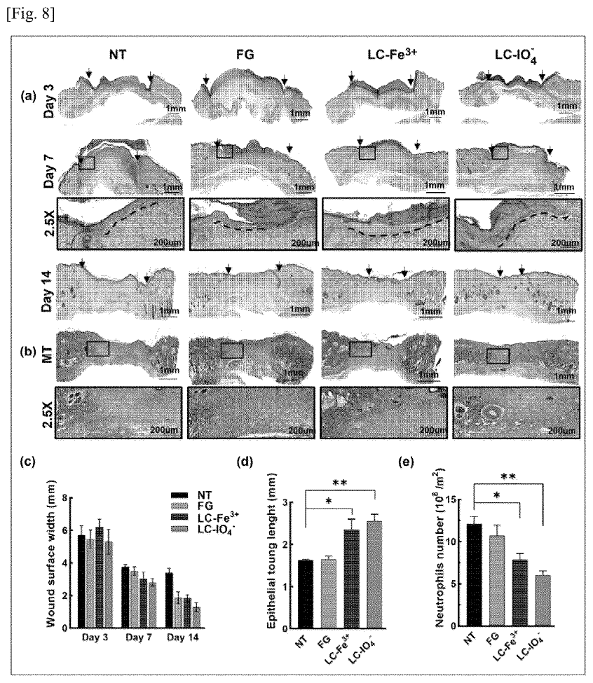

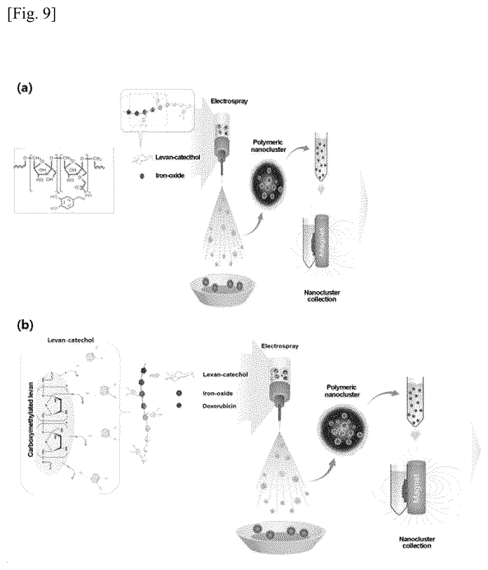

In addition, the regenerated rat tissue was histologically examined 3, 7, and 14 days after incision to further investigate the abilities of the hydrogels to accelerate wound-healing, and the results thereof were shown in A of FIG. 8. Numerous neutrophils having inflammation were observed in the nontreated group (NT), and the hydrogel-treated groups showed significantly fewer neutrophils. The newly-formed epithelial tongues of the LC-IO₄⁻ and LC-Fe³⁺ groups were 2.56±0.13 and 2.36±0.20 mm in length, respectively, after 7 days, which was significantly longer than that of the NT group (1.62±0.02 mm). In addition, B of FIG. 8 shows Masson trichrome staining images that reveal that the LC-IO₄⁻ group shows more blue-violet components that represent cell nuclei. As a result, it may be confirmed that more cells are regenerated in the wound area treated with the hydrogel than in the nontreated (control) group, and through this, it may be supported that the hydrogel of the present disclosure has excellent wound healing efficacy as the wound area is reduced in a short period of time compared to the control groups.

### Experimental Example 8. Drug Release Evaluation of LC-IO-Dox Nanocluster

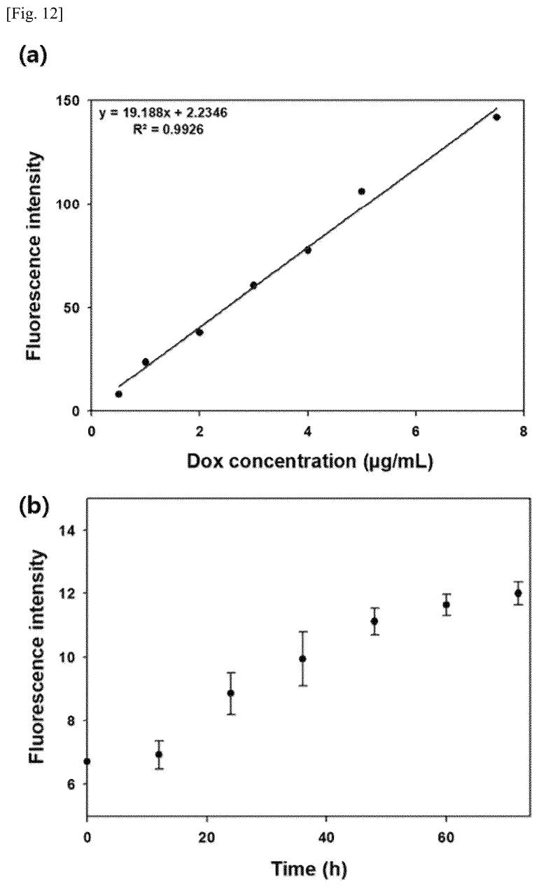

FIG. 12 shows the fluorescence intensity of Dox at an emission wavelength of 555 nm, which is confirmed for 72 hours using Levanase zymolysis of a LC-IO-Dox nanocluster containing Dox, a therapeutic drug.

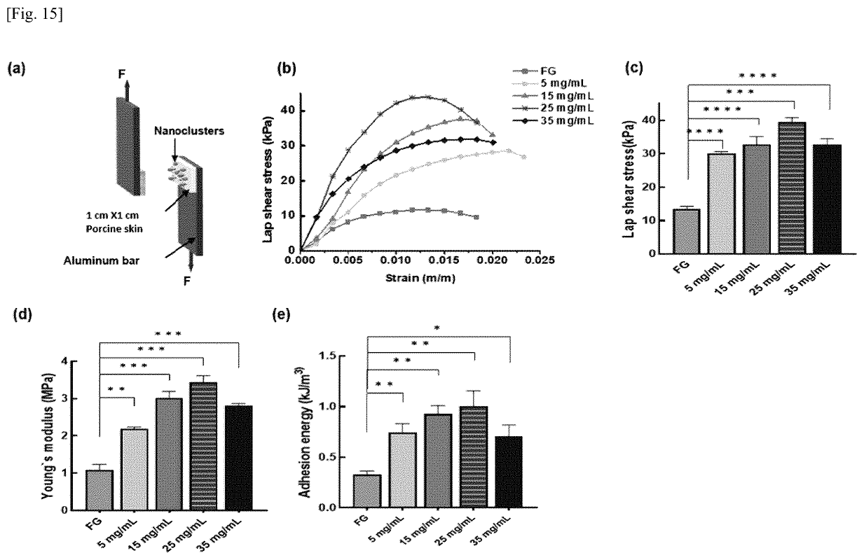

Referring to FIG. 12, a standard curve was created through an experiment in which free Dox was released (A of FIG. 12), and through this, the amount released from the LC-IO-Dox nanocluster in distilled water or 1×PBS was quantified (B of FIG. 15). Through this, it may be confirmed that Dox was well loaded into the LC-IO-Dox nanocluster, and from this, it may be confirmed that an anticancer drug Dox is released by swelling or hydrolysis of the nanocluster when applied to cancer cells through active targeting.

### Experimental Example 9. Antimicrobial Activity Evaluation of Levan, Carboxymethylated Levan, Catechol-Levan Composite, and LC-IO Nanocluster

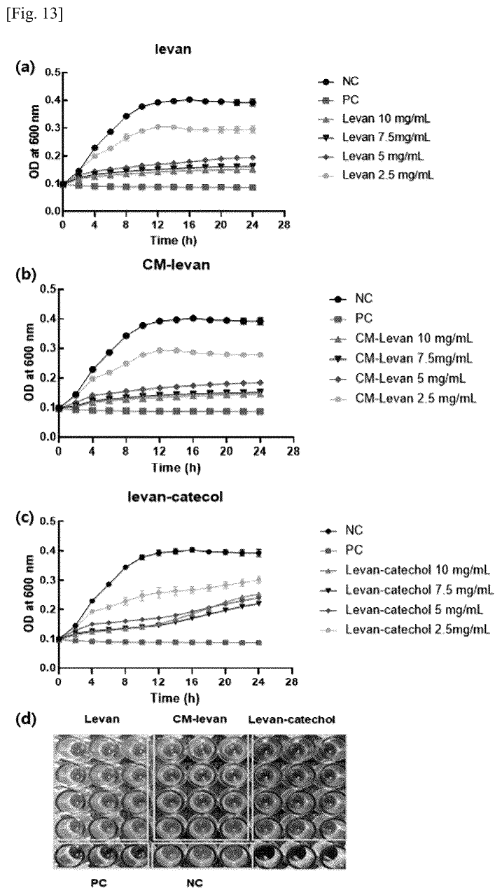

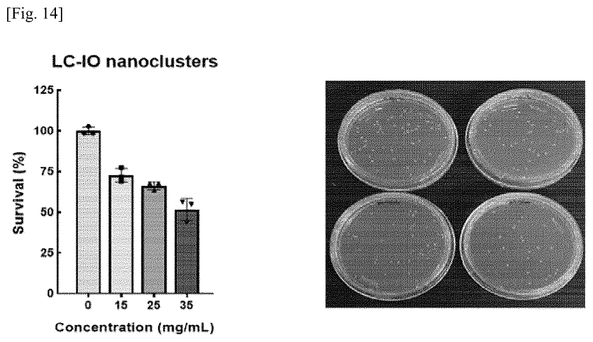

Antimicrobial activity of levan, carboxymethylated levan, a catechol-levan composite, and an LC-IO nanocluster was evaluated using E. coli, and the results of the levan, carboxymethylated levan, and catechol-levan composite were shown in FIG. 13, and the results for the LC-IO nanocluster were shown in FIG. 14 [NC: negative control, PC: positive control (Ampicilin, 1 μM)].

Specifically, the antimicrobial activity of the levan, carboxymethylated levan (CM-levan), and levan-catechol composite against E. coli was evaluated by measuring bacterial growth using OD values after treatment with different concentrations of the samples (FIG. 13). As a result, pure levan showed an inhibitory effect on the growth of E. coli after 3 hours of incubation, and the antimicrobial activity increased as the concentration increased (A of FIG. 13). The carboxymethylated levan showed an inhibitory effect on the faster growth of E. coli and showed a similar pattern of OD values according to a concentration compared to levan alone to have no significant difference (B of FIG. 13). However, in the case of the levan-catechol composite, there was no significant difference in inhibition at concentrations of 15 mg/mL or higher, and the OD values gradually increased overtime after 12 hours (not shown). This is determined due to the oxidation of the catechol group, which causes the culture medium to turn brown and interferes with the OD value (C and D of FIG. 13). Colony formation assay (FIG. 14) was performed to determine the OD value, and it was found that the number of colonies formed after treatment with the LC-IO nanocluster decreased as the concentration increased.

Meanwhile, in the case of the levan-catechol iron oxide nanocluster, the inhibition of E. coli was evaluated using only colony forming units because the color of the nanocluster interfered with the OD value (FIG. 14). As the concentration increased, bacterial viability decreased, and at 35 mg/mL, 51% of bacteria survived. However, compared with other derivatives, the nanocluster showed low inhibitory ability, which was inferred to be due to the fact that levan may be metabolized in bacterial cells. It is determined that the reason why the levan killed bacteria is due to the toxicity of the nanoparticles themselves to microorganisms, and the ability to induce osmotic stress and/or reduce water activity and compete with bacterial uptake of essential nutrients. Considering the antimicrobial activity of levan, levan is a suitable candidate for wound healing applications. Antibiotics are important for wound healing because the antibiotics help prevent or reduce bacterial infection. Wounds may provide an entry point for bacteria to proliferate and cause infection by slowing or preventing the healing process. Therefore, before the blood tissue is regenerated, it is important to supply the wound site with nutrients necessary for promoting cell growth, and preferably nutrients or metabolites that are not well utilized by microorganisms but may be utilized by human cells.

### Experimental Example 10. Tissue Adhesion Experiment of LC-IO Nanocluster Using Porcine Skin

The adhesion of LC-IO nanocluster to the biological tissue was evaluated by measuring shear stress using a universal testing machine (Instron 5544, Norwood, MA, US), and the results were shown in FIG. 15.

Referring to FIG. 15, by nanoclustering the levan-catechol composite, it is possible to improve the underwater adhesion of the levan-catechol composite and improve the storage capacity by utilizing the adhesive ability due to the large surface area of the nanocluster. In addition, the nanoclusters formed in the solution may be easily collected by magnetism and resuspended in water or 0.1 PBS and used together with a minimally invasive procedure. Lap shear stress testing was used to evaluate the adhesion ability of the nanocluster to bond two porcine skin surfaces mimicking human skin tissue (A of FIG. 15). Even at a low concentration of 5 mg/mL, the LC-IO nanocluster exhibited high adhesion strength (30.12±1.2 kPa), which was twice higher than that of commercially available fibrin glue (13.47±1.8 kPa). The adhesion strength increased with increasing concentration of nanocluster until saturation was reached and then decreased at a place where the limited adhered surface usable in a substrate was almost occupied. In addition, the nanocluster exhibited higher adhesion energy and Young's modulus than fibrin glue and showed the same pattern as shear strength.

### Experimental Example 11. Evaluation of In Vitro Hemostatic Efficacy of LC-IO Nanocluster

To evaluate the hemostatic efficacy of the LC hydrogel, a hemolysis assay was performed.

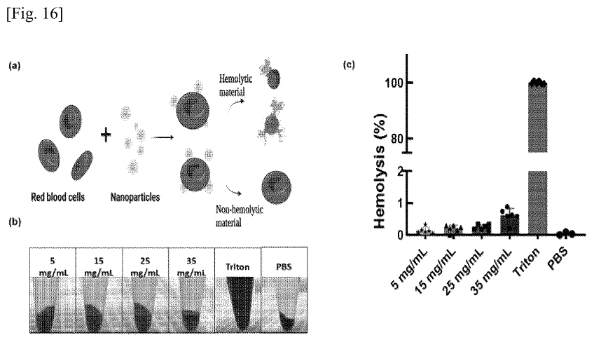

The application of inorganic nanoparticles that may cause hemolysis may limit the use as a hemostatic agent (A of FIG. 16). In order to overcome these problems, in the present disclosure, the effect of the organic nanocluster prepared according to the present disclosure on red blood cells was evaluated using a hemolysis assay (FIG. 16). The cut indicates a 5% hemolysis percentage.

The hemolysis rate was evaluated according to the method described above (Suneetha, Maduru, Kummara Madhusudana Rao, and Sung Soo Han. ACS omega 4.7 (2019): 12647-12656.). Citrated rabbit whole blood (sodium citrate solution 3.8%) was centrifuged at 4000 rpm for 10 minutes and platelet-rich plasma (PRP) was separated and red blood cells (RBCs) were washed four times with DBPS and then used for hemolysis assay. To evaluate hydrogel hemocompatibility, 500 μL of diluted RBCs (10 v/v % in DPBS 1×) were added to 100 μL of a nanocluster solution at various concentrations in a microtube and then incubated at 37° C. for 1 hour. In addition, 0.1% Triton-X and DPBS (100 μL) were used as positive and negative controls, respectively. The supernatant was collected after centrifugation at 3500 rpm for 10 minutes and analyzed using a microplate reader at 540 nm. The hemolysis rate was calculated using Equation 4 that had been described in Experimental Example 6.

Referring to B and C of FIG. 16, even at a concentration of 35 mg/mL, the LC-IO nanocluster exhibited a hemolysis percentage of less than 1%, which may confirm that the LC-IO nanocluster is compatible with the blood and a promising material for biomedical applications.

### Experimental Example 12. Evaluation of Cell Viability and Migration of LC-IO Nanocluster

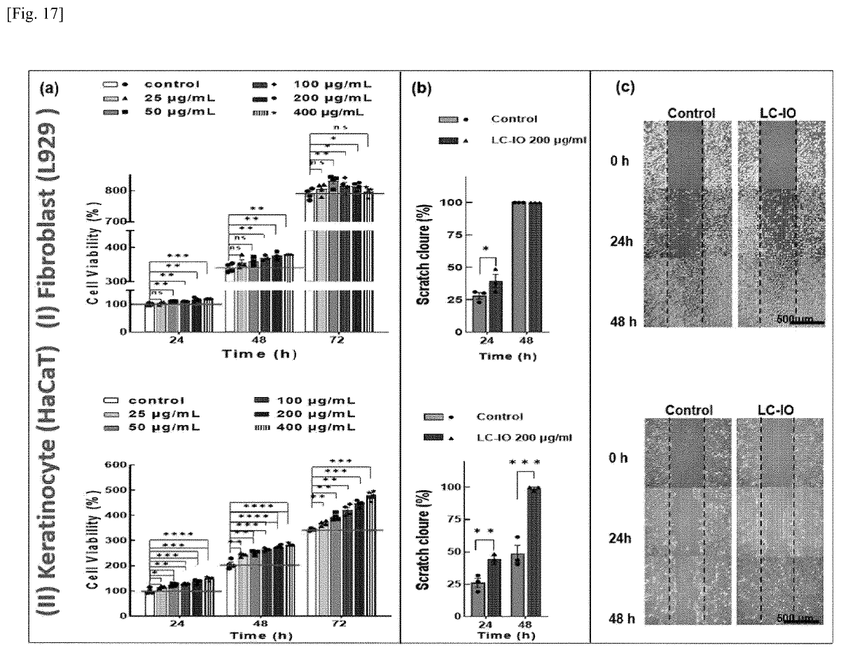

Nanocluster cytotoxicity toward L929 and HaCaT cells was assessed using the CCK-8 assay, and the results were shown in FIG. 17. The L929 cells were incubated in PRMI 1640 containing 10.0 vol % FBS and 1 vol % penicillin-streptomycin, and the HaCaT cells were incubated in DMEM containing 10.0 vol % FBS, 1 vol % penicillin-streptomycin, and 2 mM L-glutamine in a humidified incubator under 5% CO₂ conditions at 37° C. The L929 and HaCaT cells were seeded at a density of 5×10³ cells/well in a 48-well plate (200 μL) and incubated overnight at 5% CO₂ and 37° C., and then the medium was replaced with a nanocluster solution at various concentrations (25, 50, 100, 200, and 400 μg/mL). After 24, 48, or 72 hours of incubation, the culture medium was replaced with 200 μL of the culture medium mixed with a Cell Counting Kit-8 solution (10:1 v/v) and further incubated for 1 hour at 37° C. Cell viability was calculated using Equation 2 described in Experimental Example 5 above. OD values were measured at 450 nm using a microplate reader (Synergy HTX, USA).

The cell scratching assay was performed using Culture-Insert 2 wells to form a cell-free gap with 500±100 m wide before adding the nanocluster extract. The Culture-Insert 2 wells were attached to a 24-well plate and then 70 μL of cell suspensions with densities of 3×10⁵ cells/mL and 1.5×10⁵ cells/mL were added to each well and the cell culture medium filled in the outer area was incubated at 37° C. and 5% CO₂ for 24 hours. The Culture-Inset 2 wells attached with the cells were removed, and non-attached cells were washed with DPBS to be removed, and then added with a nanocluster solution. Gap closure was monitored and imaged in 24-hour intervals by optical microscopy (Optika, Italy), the scratch closure percentage was analyzed by Equation 5 described in Experimental Example 7, and three attempts were performed for each group (in Equation 5, Aₜₒ and Aₜₙ are the wound areas on day 0 and day n (n=24 and 48).

Referring to A of FIG. 17, the LC-IO nanocluster was observed to ideally affect L929 and HaCaT cell growth. For both cells, the treated LC-IO nanocluster showed increased viability with increasing concentration in the range of 25 to 400 m/mL to promote HaCaT cell growth. As a result, it may be confirmed that the LC-IO nanocluster was biocompatible by exhibiting a cell viability of 100% or higher in the range of 25 to 400 m/mL.

Next, 200 μg/mL of the LC-IO nanoclusters were applied to the L929 and HaCaT cells during the wound-scratch testing to evaluate the effect of the LC-IO nanocluster on cell migration. Referring to C of FIG. 17, it may be seen that the L929 and HaCaT cells treated with the LC-IO nanoclusters migrated much faster than control cells during co-incubation for 24 and 48 hours, and referring to B of FIG. 17, it may be seen that the LC-IO nanoclusters exhibited faster scratch closure than the control after 24 and 48 hours. The excellent cell-proliferation and fast migration data obtained for the LC-IO nanoclusters are greatly useful for wound healing applications.

### Experimental Example 13. Evaluation of In Vivo Wound Healing Ability of LC-IO Nanocluster

The in vivo wound healing ability of the LC nanocluster was evaluated using a SD rat model. All animal research was carried out in accordance with national regulations and was authorized by Postech's animal experimentation ethical committee (IRB number Postech-2022-0004).

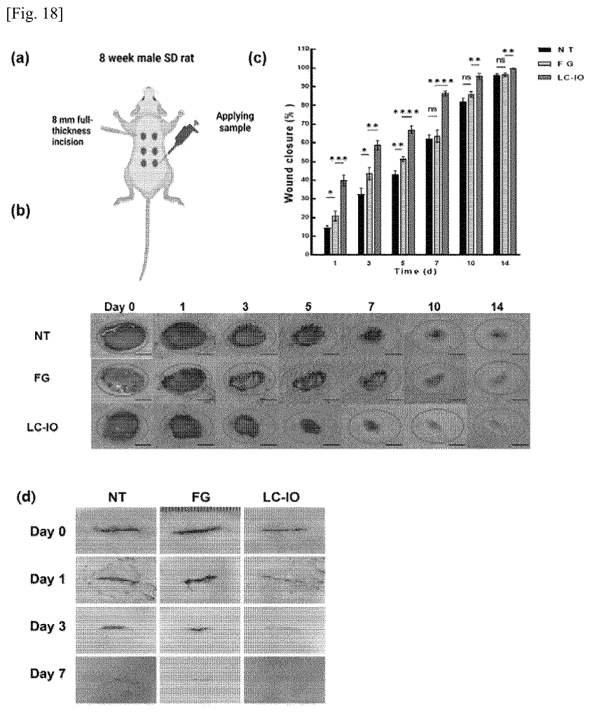

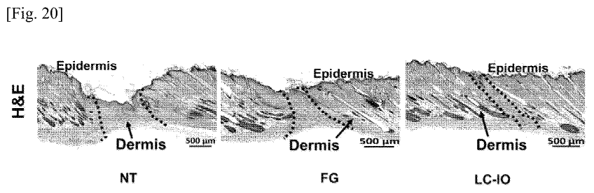

In the case of a ID skin adhesion experiment (D of FIG. 18), rats were anesthetized with isoflurane, their backs were shaved, and a narrow skin wound was made with a length of 1.5 cm and a depth of 3.0 mm. The incisions were treated with a 10 μl LC-IO nanocluster solution (25 mg/ml) and fibrin glue (FG), or nontreated (NT). The wound site was immobilized for 3 minutes while the rats were under anesthesia. Seven days after incision, the rats were euthanized, and 1 cm×2 cm skin including the wound site was excised, fixed in a p-formaldehyde solution (3.7 wt %), embedded in paraffin, and then cross-sectional sections were stained with H&E (FIG. 20).

To evaluate the bioadhesion and wound-healing properties of the LC nanoclusters, Sprague Dawley rats (SD 150 to 200 g, seven-week male) were anesthetized using isoflurane and the backs were shaved. Three 8-mm-diameter circular incisions were made on the back surface of each rat using a biopsy punch. The incisions were rapidly closed with 10 μl of a nanocluster solution (25 mg/ml) or fibrin glue (positive control), while the nontreated incision was used as a negative control (NT). The wounds were photographed on days 0, 1, 3, 5, 7, 10 and 14, and the wound closure rate (%), which was a relative wound closure (%), was calculated using Equation 5 described in Experimental Example 7 above (in Equation 5, Aₜₒ and Aₜₙ were the wound areas on day 0 and day n (n=1, 3, 7 . . . 14).

The rats were euthanized 3, 7, and 14 days after incision, and wound areas containing the wounds were excised and fixed in a p-formaldehyde solution (3.7 wt %) for subsequent hematoxylin and eosin (H&E) analysis and histological investigation using Masson's trichrome (MT) after measuring the wound size.

Referring to B of FIG. 18 showing images of skin incisions after treatment with fibrin glue (FG) and LC nanocluster samples, the wounds treated with the LC nanoclusters were closed significantly faster from the first day than fibrin glue-treated wound and untreated wound (nontreated group; NT).

Referring to D of FIG. 18 showing a photographic image of a wound adhering to nanoparticles by making a 1D wound on the back of a SD rate model to evaluate the in vivo adhesion of the LC-IO nanocluster, it may be confirmed that the wound edges smoothly adhered to each other in the wound area treated with the LC-IO nanoclusters. This is determined to be due to the small size of the nanoclusters that allow the two tissues to perfectly adhere and align with each other. In contrast, in the wound area applied with fibrin glue, the adhesive was not completely in direct contact between the two tissues in the wound area. These results mean that nanoclusters form tight macroscopic contacts to induce rapid tissue restoration when more consistent with H&E and MT staining. Referring to FIG. 20, it may be confirmed that the granulation tissue area of the LC-IO nanocluster group is narrower than that of other two groups.

Full-thickness incision of an SD rat model was used to evaluate the healing ability of the LC-IO nanocluster. B and C of FIG. 18 show skin images at various time points for wound dynamics expressed as the percentage of area healed based on a day 0 area from the first day after incision. Based on day 7, 86% of the wounds in the LC-IO nanocluster-treated group had healed, while only 63% and 6% of the fibrin glue and nontreated (NT) wounds had healed, respectively. Regenerated rat tissues were histologically investigated at 3, 7, and 14 days post-incision to further investigate the ability of nanoclusters to accelerate wound healing. The results of hematoxylin and eosin (H&E) staining were shown in A of FIG. 19, and numerous new blood vessels were observed in the groups treated with LC-IO nanoclusters 3 days after incision, which indicated faster angiogenesis that was important for wound healing. Newly formed blood vessels contribute to the formation of temporary granulation tissue, supporting the growth of new tissues and delivering nutrients and oxygen to the expanding tissue required to support the healing process. Without sufficient angiogenesis, wounds may take longer to heal and may be more susceptible to infection. In addition, the angiogenesis process helps remove waste products and dead cells from the wound site, thereby further aiding the healing process. In addition, wounds treated with the LC-IO nanoclusters showed faster re-epithelialization, and from day 3, newly formed epithelial tongues were observed only in the groups treated with LC-IO nanoclusters. Growth and migration, and epithelialization are important steps in the wound healing process because they involve the migration and proliferation of epithelial cells to cover the wound surface and restore the protective barrier function of the skin. This process is important for preventing infection, promoting tissue regeneration, and maintaining homeostasis. Restoration of the epidermis through epithelialization is a characteristic of successful wound healing. The wound surface width in the nanocluster group was shorter than that in the fibrin glue and nontreated groups.

Masson's trichrome stain (MT) is a type of histochemical stain used to determine the distribution of collagen in regenerative tissue. Keratin and muscle fibers are stained red, collagen is stained blue, the cytoplasm is stained light red or pink, and the nuclei are stained dark brown or black. The deeper the blue, the more mature the collagen is contained. MT results on day 14 (B of FIG. 19) show that treatment with the LC-IO nanoclusters not only closes the wound but also remodels the granulation tissue more rapidly into a more mature collagen content structure. A higher collagen index may indicate a greater number of fibroblasts. This is because the fibroblasts play a role in producing and constructing collagen fibers.

### Experimental Example 14. Analysis of Differentially Expressed Genes (DEGs) in In Vitro Cell Lines of LC-IO Nanoclusters

To understand the in vivo & In vitro wound healing ability of LC nanoclusters, the human genome, of which the standard genome was completed through RNAseq analysis, was utilized and Differentially Expressed Genes (DEGs) was performed to identify genes involved in human wound healing by LC-IO nanoclusters by targeting humans for wound healing. HMEC01, a human vascular cell line, and HDfn, a human skin fibroblast cell, were incubated in PRMI 1640 containing 10.0 vol % FBS and 1 vol % penicillin-streptomycin, and incubated in the presence of LC-IO nanoclusters at 37° C. in a humid incubator with 5% CO₂ conditions. After discarding the medium after incubation, the cells were placed in 1 mL of trizol, rapidly frozen at −70° C., and then tested and analyzed by ROKIT Genomics Co., Ltd., and the results were attached in Tables 1 and 2 below. Referring to Tables 1 and 2 below, it may be confirmed that genes mainly related to angiogenesis and wound healing were overexpressed compared to the LC-IO nanocluster treated group.

Lengthy table referenced here
US20260108656A1-20260423-T00001
Please refer to the end of the specification for access instructions.

Lengthy table referenced here
US20260108656A1-20260423-T00002
Please refer to the end of the specification for access instructions.

It was confirmed that the levan-catechol composite (LC) and the LC hydrogel and LC nanocluster containing the same according to the present disclosure exhibited adhesion to wet porcine skin that was about three times better than that of pure levan and a commercially available fibrin glue adhesive. In addition, the levan-catechol composite showed a low immune response close to the negative control due to low levels of endotoxin after synthesis, so that it was confirmed that the LC hydrogels and LC nanoclusters containing levan-catechol are biocompatible. In addition, it was confirmed that the LC hydrogels and LC nanoclusters exhibited excellent tissue adhesion, hemostatic properties, and wound healing promotion effects and thus may be applied as potential alternative wound dressings and tissue adhesives for bleeding control and wound treatment. In addition, it was confirmed that the LC nanoclusters according to the present disclosure may be applied as a drug delivery system by loading a drug release function.

The foregoing detailed description illustrates the present invention. Further, the aforementioned contents show and describe the preferred exemplary embodiment of the present invention, and the present invention may be used in various other combinations, modifications, and environments. That is, the foregoing content may be modified or corrected within the scope of the concept of the invention disclosed in the present specification, the scope equivalent to that of the disclosure, and/or the scope of the skill or knowledge in the art. The foregoing exemplary embodiment describes the best state for implementing the technical spirit of the present invention, and various changes required in specific application fields and uses of the present invention are possible. Accordingly, the detailed description of the invention above is not intended to limit the invention to the disclosed exemplary embodiment. Further, the appended claims should be construed to include other exemplary embodiments.

LENGTHY TABLES
The patent application contains a lengthy table section. A copy of the table is available in electronic form from the USPTO web site (https://seqdata.uspto.gov/docdetail?docId=US20260108656A1). An electronic copy of the table will also be available from the USPTO upon request and payment of the fee set forth in 37 CFR 1.19(b)(3).

1. A levan-catechol composite comprising carboxymethylated levan and dopamine.

2. The levan-catechol composite of claim 1, wherein the carboxymethylated levan comprises a repeating unit represented by the following Chemical Formula 1: 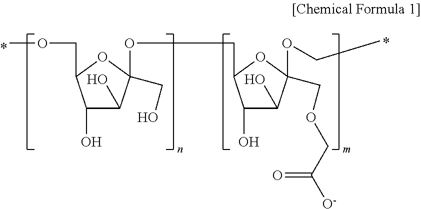
wherein the n and m are mole fractions within the repeating unit, respectively, wherein 0.7≤n≤0.95, 0.05≤m≤0.3, and n+m=1.

3. The levan-catechol composite of claim 1, wherein the levan-catechol composite comprises a repeating unit represented by the following Chemical Formula 2: 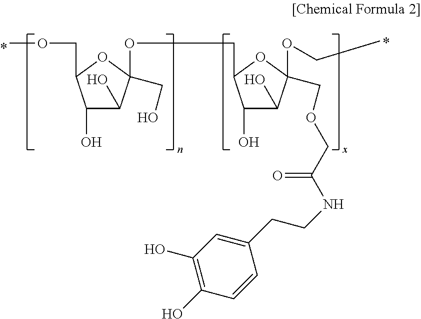
wherein the n and x are mole fractions within the repeating unit, respectively, wherein 0.7≤n≤0.95, 0.05≤m 0.3, and n+x=1.

4. The levan-catechol composite of claim 1, wherein the weight ratio of the carboxymethylated levan and dopamine is 1:0.5 to 0.9.

5. A tissue adhesion composition comprising the levan-catechol composite according to claim 1.

6. The tissue adhesion composition of claim 5, further comprising:
ferric chloride, sodium periodate, horseradish peroxidase and hydrogen peroxide, fibrinogen or a mixture thereof as a cross-linking agent.

7. The tissue adhesion composition of claim 5, wherein the tissue adhesion composition is used for hemostasis, wound healing or closure.

8. A method for preparing a levan-catechol composite comprising:
(a) carboxymethylating levan to obtain carboxymethylated levan; and
(b) conjugating the carboxymethylated levan to dopamine through EDC-NHS coupling.

9. A nanocluster comprising the levan-catechol composite according to claim 1; and a hydrophobic material.

10. The nanocluster of claim 9, wherein the nanocluster has a core-shell form including a core containing the hydrophobic material; and a shell surrounding the core and containing the levan-catechol composite.

11. The nanocluster of claim 9, wherein the average diameter of the nanocluster is 1 to 600 nm.

12. The nanocluster of claim 9, wherein the hydrophobic material comprises magnetic nanoparticles, metal nanoparticles, drugs, or a combination thereof.

13. The nanocluster of claim 12, wherein the magnetic nanoparticles are at least one selected from iron (II) oxide, iron (III) oxide, cobalt ferrite, zinc ferrite, nickel ferrite, manganese ferrite, iron, cobalt, nickel, manganese, FeAu, FePt and CoNi,
the metal nanoparticles are at least one selected from gold, silica, titania and magnesium, and
the drugs are at least one material selected from the group consisting of hydrophobic drugs, nucleic acids, proteins, polypeptides, carbohydrates, inorganic materials, antibiotics, anticancer agents, antimicrobial agents, steroids, anti-inflammatory analgesics, sex hormones, immunosuppressants, antiviral agents, anesthetics, antiemetics, antihistamines, local anesthetics, antiangiogenic agents, vasoactive agents, anticoagulants, immunomodulators, cytotoxic agents, antibodies, neurotransmitters, psychotropic drugs, oligonucleotides, lipids, cells, tissues, cancer chemotherapeutic agents and vaccines.

14. A tissue adhesion composition comprising the nanocluster according to claim 9.

15. The tissue adhesion composition of claim 14, wherein the nanocluster comprises iron oxide nanoparticles as the hydrophobic material.

16. A drug delivery system comprising the nanocluster according to claim 9.

17. The drug delivery system of claim 16, wherein the nanocluster is a hydrophobic material, and comprises a magnetic nanoparticle-drug composite formed by conjugating the magnetic nanoparticles and the drug.

18. A method for preparing a nanocluster, comprising (A) electrospraying a solution for preparing a nanocluster, in which the levan-catechol composite according to claim 1 and a hydrophobic material are dispersed, into an aqueous solution.

19. The method for preparing the nanocluster of claim 18, wherein the solution for preparing the nanocluster is obtained by mixing a first dispersion solution in which the levan-catechol composite is dispersed in a first solvent; and a second dispersion solution in which the hydrophobic material is dispersed in a second solvent, and
the first solvent and the second solvent are the same as or different from each other, and each independently at least one selected from the group consisting of hexane, toluene, tetrahydrofuran (THF), dimethylformamide (DMF), dimethyl sulfoxide (DMSO), and alcohol-based compounds.

20. A tissue adhesion method comprising treating the tissue adhesion composition including the levan-catechol composite according to claim 5 to a subject in need thereof.

21. A tissue adhesion method comprising treating the tissue adhesion composition including the nanocluster according to claim 14 to a subject in need thereof.
